(19) United States
(12) Patent Application Publication
(10) Pub. No.: US 2011/0144636 A1
(43) Pub. Date: Jun. 16, 2011

(54) UNIVERSAL SURGICAL FUNCTION CONTROL SYSTEM

(76) Inventors: David Austin Alexander, Sterrett, AL (US); Rizk El-Galley, Birmingham, AL (US); Mary Hawn, Birmingham, AL (US)

(21) Appl. No.: 12/867,989

(22) PCT Filed: Feb. 18, 2009
(86) PCT No.: PCT/US09/34425

§ 371 Date: Jan. 19, 2011

Related U.S. Application Data
(60) Provisional application No. 61/029,487, filed on Feb. 18, 2008.

(51) Int. Cl. A61B 18/12

(52) U.S. Cl. 606/34

(57) ABSTRACT

A control system includes a selector by which a user can select any of a number of surgical devices or similar devices for use and a processor system responsive to user actuation of a foot control or other central control by controlling the selected device. Each device has an associated intelligent adapter that communicates information relating to device with which the adapter is associated. The processor system uses the information communicated by the adapter to properly interface the associated device with the control system and its central control.

### CROSS-REFERENCE TO RELATED APPLICATIONS

This application claims benefit of U.S. Provisional Application No. 61/029,487, filed Feb. 18, 2008, which is hereby incorporated herein by reference in its entirety.

### BACKGROUND OF THE INVENTION

1. Field of the Invention

The present invention relates generally to electrosurgical devices and, more specifically, to controlling multiple electrosurgical devices from a single controller.

2. Description of the Related Art

Laparoscopic surgery is increasingly common. The principle of laparoscopic surgery is to perform a surgical procedure with small keyhole incisions. Usually, two or three such keyhole incisions are made in the abdomen for insertion of a telescopic video camera, laparoscopic instruments and electrosurgical devices. Electrosurgical devices are used in both open surgical and laparoscopic surgical procedures to cut and coagulate tissue. Various types of electrosurgical devices are known, including those that use diathermy with either unipolar or bipolar current, and advanced devices such as harmonic scissors and argon beam and laser devices. Monopolar and bipolar devices use one or two electrodes, respectively, to deliver electrical energy from a current source to the surgical site. By varying the voltage, current, or waveform of the electrical energy delivered by the electrode, surgeons can cut tissue cleanly, coagulate tissue to stop bleeding, or produce a “blended cut” that combines these two functions.

A surgeon may use more than one electrosurgical device in a major surgical procedure. The surgeon operates each device independently of the others, typically using a foot pedal control connected to the device. Thus, the surgeon may have at his or her feet several foot pedal controls, each for operating a different device. Multiple foot pedal controls on the floor beneath the operating table create the potential for confusion and increased risk of injury when the surgeon looks under the table to locate the foot pedal control associated with the particular electrosurgical device he or she intends to use, thereby losing sight of the surgical field. The potential for confusion is compounded by the foot pedals of different devices having different uses or functions. For example, unipolar electrosurgical devices commonly have two foot pedals: depressing one pedal causes the device to apply a high-power signal to the electrode for cutting tissue; depressing the other pedal causes the device to apply a lower-power signal to the electrode for coagulating tissue. Bipolar electrosurgical devices most commonly have only one foot pedal, which, when depressed, causes the device to energize or apply a signal to the electrode, i.e., it turns the power on. (Releasing it de-energizes the electrode.) Some bipolar devices include a second pedal, but the functions of the two pedals of a bipolar device are different from those of unipolar devices: depressing one pedal causes the bipolar device to, as described above, turn the power on; depressing the other pedal causes the device to increase the power (proportionately to the amount of time that pedal is depressed). Thus, there is a rist of injury due to surgeon confusion arising from the differing functions associated with the foot pedals.

Additionally, because the surgeon may operate multiple electrosurgical devices independently from each other in a major surgical proceudre, there is no system to evacuate smoke when the devices perform cutting or coagulation functions. Delay evactuating smoke can cause difficulty in viewing the surgical field and may neccessiate delay in the surgery while smoke is evactuated from the surgical field. Further, in the past, sugeons would have to stop using one electrosurgical device to insert a vacuum and remove any smoke and debris, causing further delay in the surgical procedure.

Due to the lack of integration of the electrosurgical devices that produce this smoke, no system has traditionally been available that can read and react to the amount and type of energy being applied to the tissue. While some smoke evacuation systems do exist, none of them can intelligently and automatically alter the intensity or longevity of smoke evacuation based on surgical conditions or in reaction to surgical activities.”

It would be desirable to provide a control system for electrosurgical devices operated by foot pedals or similar controls that alleviates the potential for confusion and that allows for automatic and intelligent activation of a smoke evacuation system when a surgical function is performed. The system described herein addresses this problem and others in the manner described below.

### SUMMARY OF THE INVENTION

A control system is provided that allows a surgeon or other user to use a central control, such as a foot control, to operate a plurality of independent electrosurgical devices, each of which would otherwise need to be individually controlled by an associated foot control or other device control.

The control system includes a device selector by which a user can select an instrument for use. The control system also includes a processor system that is programmed or adapted to respond to user actuation of the central control by controlling the selected electrosurgical device. Because each device may have input requirements or other interface considerations that are different from those of the other devices of the plurality, an intelligent adapter is provided for each device. Each adapter is programmed or adapted to communicate information relating to the device with which it is associated. The processor system uses the information communicated by the adapter to properly interface the associated device with the control system and its central control. Thus, for example, in exemplary embodiments of the invention, a surgeon can use a central foot control to control any selected one of a number of electrosurgical devices connected to the control system that would otherwise need to be controlled by a corresponding number of individual foot controls.

It is to be understood that both the foregoing general description and the following detailed description are exemplary and explanatory only and are not restrictive of the invention, as claimed.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 is a schematic view of one embodiment of a control system for electrosurgical and other devices;

FIG. 2 is a block diagram of one embodiment of a controller unit of the control system;

FIG. 3 is a schematic view of one embodiment of an intelligent adapter of the control system;

FIG. 3A is a schematic view of one embodiment of an intelligent adapter of the control system for a “smart” electrosurgical device;

FIG. 4 is a block diagram of the intelligent adapter;

FIG. 5 is a schematic view of one embodiment of a remote controller of the system for selecting devices and related functions;

FIG. 6 shows the remote controller enclosed in an anti-static sheath;

FIG. 7 is an enlargement of a portion of FIG. 6 and shows the sheath cinched around an electrically conductive portion of the remote controller cable for bleeding off static charge;

FIG. 8 is a schematic view of another embodiment in which the devices themselves have inputs for selecting them;

FIG. 9 shows an exemplary screen display produced by the system;

FIG. 10 shows another exemplary screen display produced by the system;

FIG. 11 shows a further exemplary screen display produced by the system;

FIG. 12 is a flow diagram illustrating a method by which one embodiment of the system operates;

FIG. 13A is a flow diagram illustrating a portion of the method of FIG. 12;

FIG. 13B is a continuation of the flow diagram of FIG. 13A;

FIG. 13C is a continuation of the flow diagram of FIGS. 13A-B;

FIG. 13D is a continuation of the flow diagram of FIGS. 13A-C;

FIG. 13E is a continuation of the flow diagram of FIGS. 13A-D;

FIG. 14 is a schematic view of one embodiment of a remote controller;

FIG. 15 is a schematic view of prior art connections of ESU's to respective foot controls;

FIGS. 16A-D are schematic views of alternative embodiments of the control system;

FIG. 17 is a block diagram of one embodiment of a controller unit of the system;

FIG. 18 is an exemplary front elevational view of an assembled system of a movable cart;

FIG. 19 is an exemplary rear elevational view of an assembled system of FIG. 18, showing a plurality of intelligent interfaces connecting the respective ESU's and the central controller unit;

FIG. 20 are side elevational views of connections between a prior art ESU/foot control and an ESU and intelligent interface;

FIG. 21 is an exemplary embodiment of a front panel of a central controller unit of the control system;

FIG. 22 is an exemplary embodiment of a rear panel of a central controller unit of the control system;

FIG. 23 are schematics showing a conversion circuit and an EEPROM memory cell in an exemplary embodiment of an intelligent interface;

FIG. 24 is a schematic circuit of an exemplary conversion circuit for an Ethicon Hoimonic Scalpel device;

FIG. 25 is a schematic circuit of an exemplary conversion circuit for an Olympus SonoSurg device;

FIG. 26 is a schematic circuit of an exemplary conversion circuit for controlling devices with either analog or digital input requirements, or both.

FIG. 27 is a schematic circuit of an exemplary digital translator module.

FIG. 28 is a schematic circuit of an exemplary analog translator module.

FIGS. 29-32 are exemplary schematic views of optional aspects of the control system;

FIG. 33 is a schematic view of an exemplary bypass circuit of the control system;

FIGS. 34-36 are exemplary elevational views of alternative embodiments of a central user control, showing a left and right switch as well as a device select switch;

FIGS. 37-41 are exemplary display screen images, showing the selected device and the operations of the respective pedals of the central user control, and showing, in FIG. 41, the smoke evacuation system in an “ON” position during a “CUT” operation;

FIGS. 42-45 are exemplary top elevational views of alternative embodiments of a central user control;

FIGS. 46-49 are exemplary display screen images, showing the selected device and the operations of the respective pedals of the central user control;

FIG. 50 is an alternative embodiment of a front panel of a central controller unit of the control system, showing a internal display positioned therein the front panel;

FIGS. 51-53 show exemplary displays of the front panel of the central controller of FIG. 50; FIG. 51 shows a unipolor device selected and the respective left control is “CUT” and the center control is “COAG”; FIG. 52 shows a surgical drill selected and the left control is the drill ON/OFF control and the other controls are not used; and FIG. 53 shows a selected drill device in which the ON/OFF control is actuated and active and the “green” LED light on the left control indicator is blinking;

FIGS. 54-59 show exemplary block diagrams of one embodiment of the control system that is adapted to interface with a Bluetooth wireless foot control device; FIG. 54 shows a general block diagram using the Bluetooth wireless foot control and shows optional aspects including visual and voice processing circuitry; FIG. 55 shows a general block diagram using a general foot control with the optional aspects including visual and voice processing circuitry; FIGS. 56 and 57 show rear panel views configured for use with the Bluetooth wireless foot control and the general foot control respectively; FIG. 58 is a block diagram of one embodiment of the visual guidance circuitry and FIG. 59 is a block diagram of one embodiment of the voice guidance circuitry;

FIG. 60 is a schematic view of the control system suspended from the ceiling of an operating room;

FIG. 61 shows a schematic view of an exemplary integrated operating room control system that could comprise the electrosurgical and linear devices, camera/video control, insufflator control, and smoke evacuation control; and

FIGS. 62-65 are exemplary display screen images for the integrated operating room control system, showing the selected device and the operations of the respective pedals of the central user control.

### DETAILED DESCRIPTION OF THE INVENTION

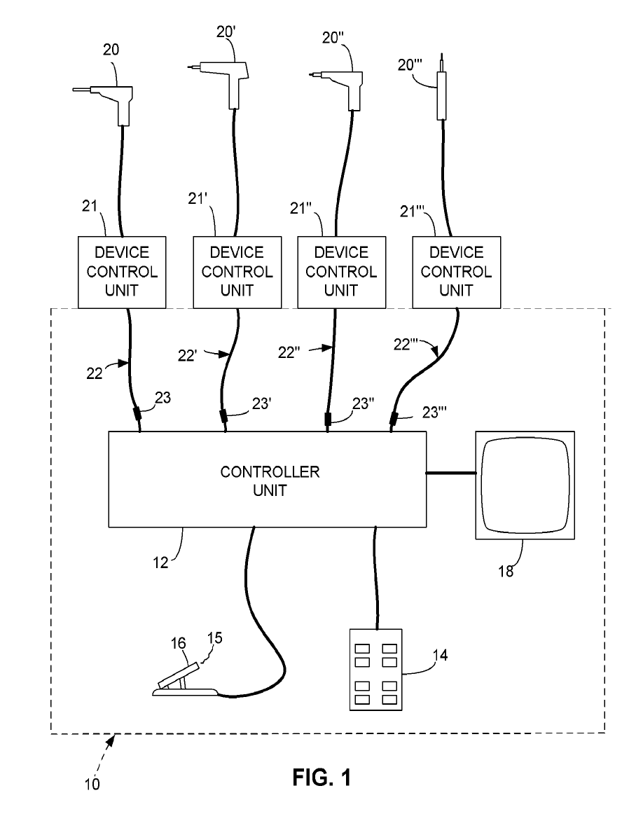

As illustrated in FIG. 1, an electrosurgical control system 10 includes a central controller unit 12, a device selector such as a remote controller 14, a central user control 15 such as a foot control 16, and a display 18 that can be either a dedicated display or monitor for the purposes described below or, in some embodiments of the invention, can be the monitor that displays laparoscopic video imagery for a surgeon. System 10 is shown in FIG. 1 as, for exemplary purposes, controlling four electrosurgical tools or instruments 20, 20′, 20″ and 20′″ via their associated device control units 21, 21′, 21″ and 21′″, but in other embodiments can control any suitable number and type of such instruments. Each of instruments 20, 20′, 20″ and 20′″ is controlled by and communicates with control system 10 via a channel. Thus, in the illustrated embodiment, control system 10 has a first, second, third and fourth channel, but in other embodiments can have more or fewer channels.

The term “device” or “electrosurgical device” is used in this patent specification to refer to not just the instrument (e.g., 20, 20′, 20″ and 20′″) itself, but rather, if the instrument is usable in combination with a control unit (e.g., 21, 21′, 21″ and 21″') that may be conventionally associated with the instrument, to the combination of the instrument and its associated control unit. In other words, an “electrosurgical device” comprises the electrosurgical instrument (e.g., 20, 20′, 20″ and 20″') and its device control unit (e.g., 21, 21′, 21″ and 21′″, respectively) that are conventionally intended to be used together or sold together commercially as a unitary product. Thus, it is the instrument and its associated control unit that are well-known in the art and commercially available but with which the control system 10 can be used in combination as described in this patent specification. In addition, when an electrosurgical device is obtained commercially, although not shown in FIG. 1 for purposes of clarity, an associated foot control (much like foot control 16) or other device user control can be connected directly to the associated device control unit. As known in the art, by depressing the pedals of such a device user control, the surgeon or other user can operate the associated one of electrosurgical instruments 20, 20′, 20″ and 20′ in the manner known in the art.

Electrosurgical instruments 20, 20′, 20″ and 20′″ and others like them can be of any suitable type known in the art, including those that use diathermy with either unipolar or bipolar current (commonly referred to simply as unipolar devices and bipolar devices), and advanced devices such as harmonic scissors and argon beam and laser devices. The illustrated shapes and other structural features of instruments 20, 20′, 20″ and 20′″ as depicted in FIG. 1 are not intended to describe the instruments specifically but rather are intended only to convey the general concept that various instruments can be used. Indeed, it is important to note that the described system facilitates the integration of instruments that may have different functions and other characteristics in terms of how they respond to their associated device user controls (not shown) and in terms of the signals produced by their device user controls that characterize their operation. For example, instruments 20, 20′ and 20″ can have functions that differ from those of each other as a result of device being, for example, a unipolar device, while device′ is, for example, a bipolar device, and device″ is a harmonic device. In addition, it may be that, for example, instruments 20 and 20′ have different operating characteristics from each other because they require signals of different voltages from each other. The various devices may be produced by different manufacturers or be different versions or models of a device. Regardless of any such differences, control system 10 ensures that any and all of the instruments to which it is connected can be controlled by foot control 16 or other central user control.

Control system 10 further includes intelligent adapters 22, 22′, 22″, and 22′″, each associated with one of instruments 20, 20′, 20″ and 20′″, respectively. Each of intelligent adapters 22, 22′, 22″ and 22′″ includes a suitable cable and may include an adapter module 23, 23′, 23″ and 23′″, respectively, which comprises an enclosure for the intelligent electronics described below that are programmed or adapted to interface foot control 16 with a user-selected one of instruments 20, 20′, 20″ and 20′″ as described in further detail below. The intelligent adapters 22, 22′, 22″, and 22′″ include an adapter module 23, 23′, 23″ and 23′″ when the device, 20′, 20″ and 20′″ is a “dumb” instrument, i.e., one that has no computer communication port. When the instrument is a “smart” instrument i.e., one that has a computer communication port and intelligent electronics, then the intelligent adapter 22, 22′, 22″, and 22′″ may include only a cable. Thus, for example, although absent control system 10, a surgeon would have to use four separate foot controls (not shown), each associated with one of instruments 20, 20′, 20″ and 20′″, by using the control system 10, the surgeon can select any one of instruments 20, 20′, 20″ and 20′″ and use foot control 16 to control it. By making such selections from time to time as needed during a surgery, the surgeon can readily use any or all of instruments 20, 20′, 20″ and 20′″ without moving from foot control 16 and without diverting his or her eyes from the surgical field.

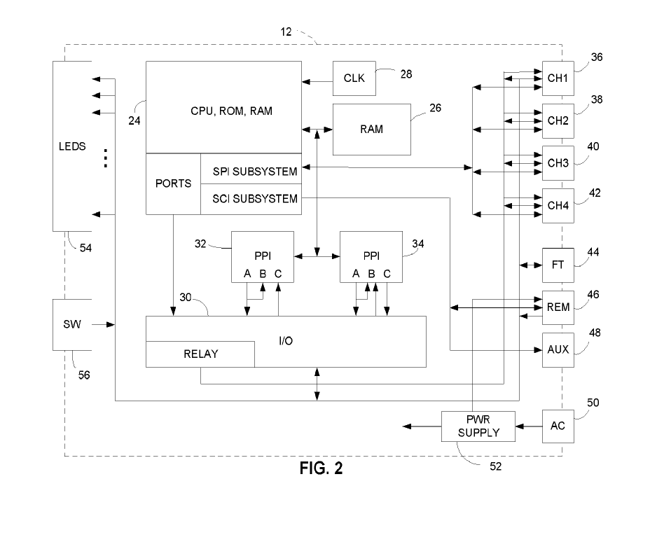

As illustrated in FIG. 2, in an exemplary embodiment of the invention, central controller unit 12 includes, within a suitable electronics enclosure or housing (not shown), a processor system having a microcontroller 24 with a central processing unit (CPU) that is programmed to affect the method steps described below. The programming can be stored in suitable read-only memory (ROM). Suitable random-access memory (RAM) 26 is also included to enable proper operation of the CPU. These memories can be integrally formed in microcontroller 24 along with the CPU and other portions generally included in microcontrollers and microprocessors or can be external to it in other embodiments. The MC68HC711E20, available from Motorola, is an example of a suitable microcontroller 24. A system clock 28 is also included to enable proper operation of microcontroller 24. In view of the description below of the method steps, persons skilled in the art will be capable of providing suitable programming and otherwise configuring and using central controller unit 12.

Ports of microcontroller 24 are coupled to input/output (I/O) circuitry 30, as are two programmable peripheral interfaces (PPIs) 32 and 34. The 82C55, available from OKI Semiconductor, is an example of a suitable PPI. Input/output circuitry 30 interfaces the above-described logic with channel connectors 36, 38, 40 and 42, a foot pedal connector 44, and a remote unit connector 46. Other connectors on or in the enclosure include an auxiliary data connector 48, to which a computer (not shown), a display, or other external equipment can be connected, and an AC power connector 50 through which central controller unit 12 receives power to operate its circuitry and, via remote connector 46, the circuitry of remote controller 14. A power supply circuit 52 distributes the power to such circuitry.

A computer connected to auxiliary data connector 48 can include display 18 (see FIG. 1), although such a computer is not shown in FIGS. 1 and 2 for purposes of clarity. As noted above, display 18 can be that of such a computer or can be the very laparoscopic monitor used in the surgery in which the system is used. As described below in further detail, a surgeon can view the monitor not only to view the laparoscopy but also to view information output by central controller unit 12. This information can be superimposed on the laparoscopic image, located in a corner of display 18 or otherwise located in a convenient position and manner on display 18. The requisite laparoscopic equipment, including its monitor or display, is well-known in the art and not illustrated in this patent specification for purposes of clarity but is present in instances in which an embodiment of the invention is used in laparoscopic surgery. Video combiner circuitry to superimpose information output by central controller unit 12 over laparoscopic imagery is not show for purposes of clarity, but suitable circuitry is well-known and commercially available.

Input/output circuitry 30 also interfaces the above-described logic with a number of suitable display elements, such as light-emitting diodes (LEDs) 54. LEDs 54 can indicate to a user, in addition to system status and error conditions, such as whether power is on, etc., whether any electrosurgical devices have been connected to connectors 36, 38, 40 and 42 and, if so, which one of them a user may have selected. Such indications are similar to those described below with regard to remote controller 14. Input/output circuitry 30 also interfaces the above-described logic with a device select switch 56 that, as described in further detail below, a user can use to select one of the connected electrosurgical devices as an alternative to using remote controller 14.

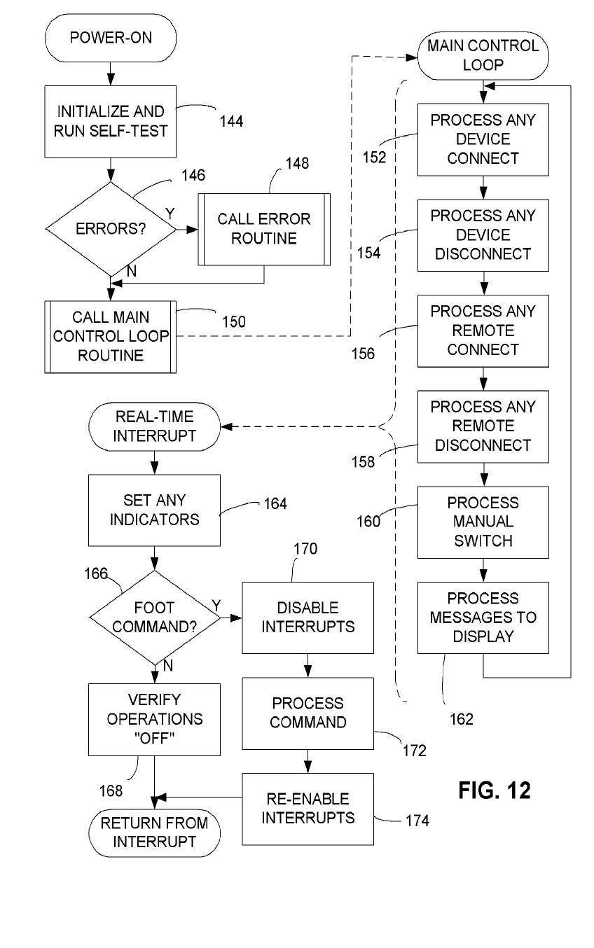

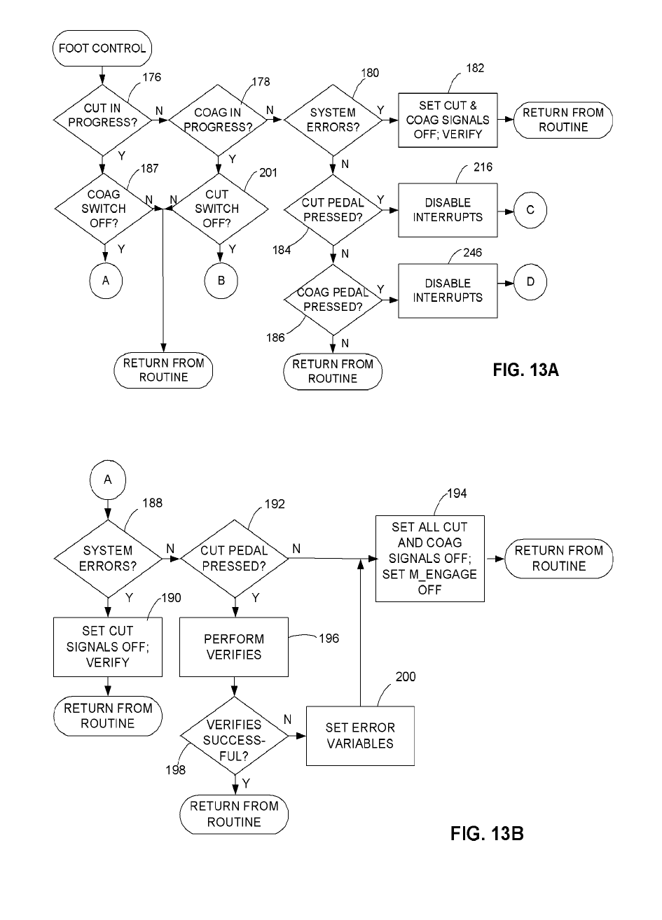

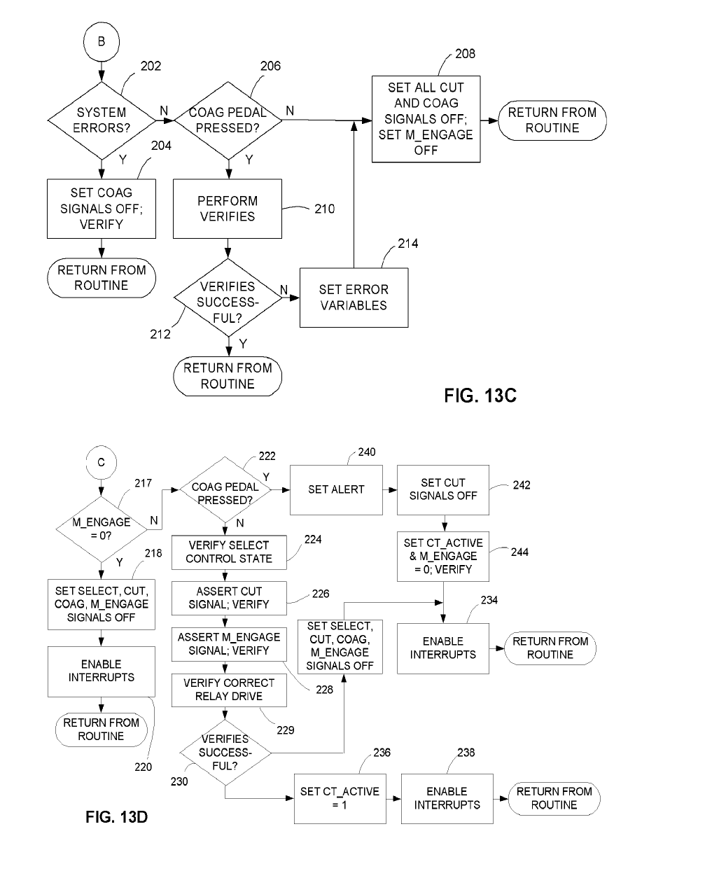

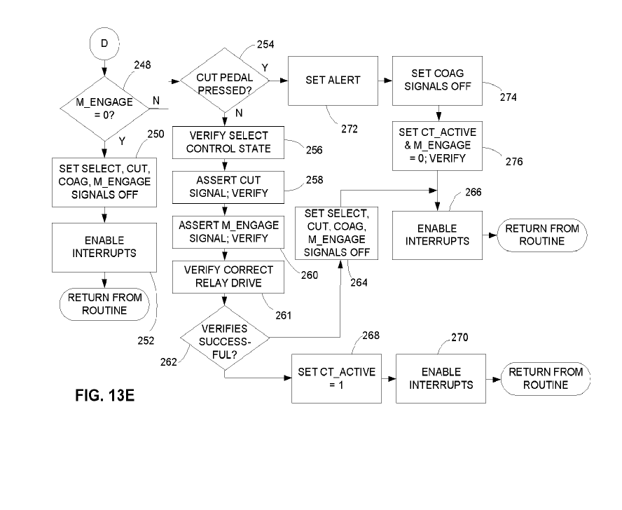

Functions of PPIs 30 and 32 are indicated below with regard to FIGS. 12 and 13A-E, which illustrate the method by which central controller unit 12 operates. The signals to which the relevant method steps relate can optionally include, as indicated in FIG. 2: signals received at port C of PPI 32 from device select switch 56; signals received at port C of PPI 32 from channel connectors 36, 38, 40 and 42 that indicate whether a device is connected; signals received at port C of PPI 32 from foot pedal connector 44 that indicate the state of each foot pedal (i.e., depressed or not depressed); signals generated at port A of PPI 32 that are provided to electrosurgical devices connected at connectors 36, 38, 40 and 42; signals received at port B of PPI 32 that read or “verify” the signal level provided to electrosurgical devices at connectors 36, 38, 40 and 42; signals generated at port C of PPI 34 that are provided to LEDs 54; signals generated at port A of PPI 34 that are provided to relay drive circuits in I/O circuitry 30 to enable signals to reach a (selected) electrosurgical device at connectors 36, 38, 40 and 42; signals received at port B of PPI 34 that read or “verify” the signal level provided to the relay drive circuits; signals received at port B of PPI 34 from connector 44 that indicate whether foot control 16 is connected; and signals received at port B of PPI 34 from connector 46 that indicate whether remote controller 14 is connected.

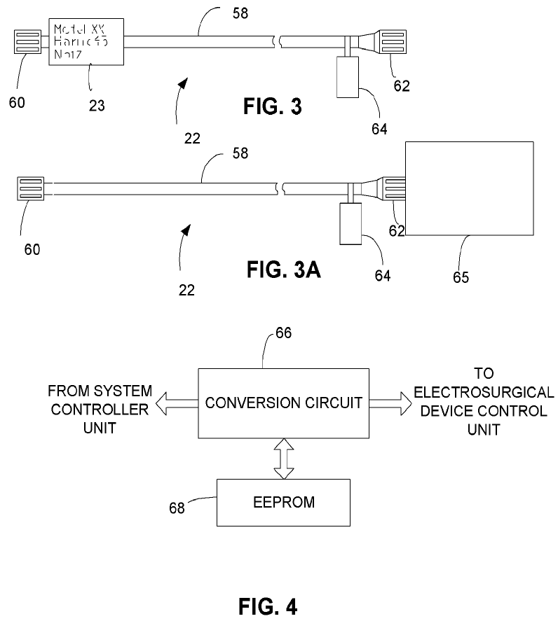

As illustrated in further detail in FIG. 3, each intelligent adapter (e.g., 22) includes, in addition to a suitable length of cable 58, the adapter module (e.g., 23) that houses the intelligent logic described below, and two adapter connectors 60 and 62. In preparation for use, a user can connect adapter connector 60 to any one of channel connectors 36, 38, 40 and 42, and connect adapter connector 62 to its associated device control unit (e.g., 21).

Alternatively, if a “smart” electrosurgical device 65 is used, the intelligent adapter 22 may not include an adapter module 23, as illustrated in FIG. 3A. In this embodiment, the electrosurgical device houses the intelligent logic described below.

As illustrated in FIG. 4, adapter module 23 can comprise an embedded microchip conversion circuit 66 (providing “intelligence” in according with its programming) and a memory 68, such as an electrically eraseable programmable read-only memory (EEPROM), from which central controller unit 12 can read information relating to the electrosurgical device associated with that intelligent adapter. Alternatively, if the electrosurgical device is “smart” the device may include an embedded microchip conversion circuit 66 and a memory 68, from which central controller unit 12 can read information relating to the electrosurgical device associated with that intelligent adapter. The information can include information identifying functions of the electrosurgical device, such as whether a foot pedal is used for activating a cutting function or a coagulation function, for turning the device on and off, or for another function. The information can include information identifying the device type, e.g., unipolar, bipolar, harmonic scissors, argon beam, etc. The information can include information identifying the manufacturer name and model number or other identifying information that may aid the user. The information can include information that characterizes the operation of the device user control (e.g., foot pedal) that is conventionally associated with the electrosurgical device. If the electrosurgical device is “smart,” the information may also include information regarding the power level and adjustments thereto, unit diagnostics, and the like. Central controller unit 12 can use such information to conform the signals it provides to the electrosurgical device to the parameters under which that device conventionally operates, i.e., conventionally would receive from its associated device user control if such a device user control were connected. As indicated in FIG. 3, some of this information, such as the device type and manufacturer name and model number can be imprinted on module 23 where it can be read by a user. Similarly, such information can be imprinted on a hanging tag 64 attached to an end of the cable.

It is of course contemplated that all of the relevant intelligent logic can be stored within each intelligent adaptor. Alternatively, a portion of the intelligent logic can be stored within each intelligent adaptor and the remainder can be stored within the central controller unit 12. In this aspect, for example and without limitation, the intelligent adaptor can only have a stored identification number that can be used to point to the full database for that respective device that is stored within the central control unit.

In another aspect, the intelligent adaptor 22 can comprise an adaptor module and two adaptor connectors 60, 62. In this aspect, a user can connect the adaptor connector 60 to any one of the channel connectors and connect adapter connector 62 to its associated device control unit. In this aspect, it is contemplated that the adaptor module 23 simple comprises conversion electronic circuitry that is configured so that the adaptor connector 60 for each of the intelligent adaptors 22 can be uniform—thus allowing for the use of the common channel connectors on the central control unit 12. The conversion circuitry converts the manufacturer's presumably non-standard connector to a form that can be readily implemented in the adaptor connector 60. In this example, it is contemplated that the remaining intelligent logic would be present in memory that is coupled to the central controller unit 12. In one example, the memory could be EEPROM that is located within the central controller unit.

With further regard to FIG. 4, in the exemplary embodiment of the invention, conversion circuit 66 converts input control signals received from central controller unit 12 to emulate the mechanical or solid-state switch closures of a foot pedal or similar switch-based device user control. As described below in further detail, memory 68 clocks bits out serially to central controller unit 12 in response to a clock signal received from central controller unit 12.

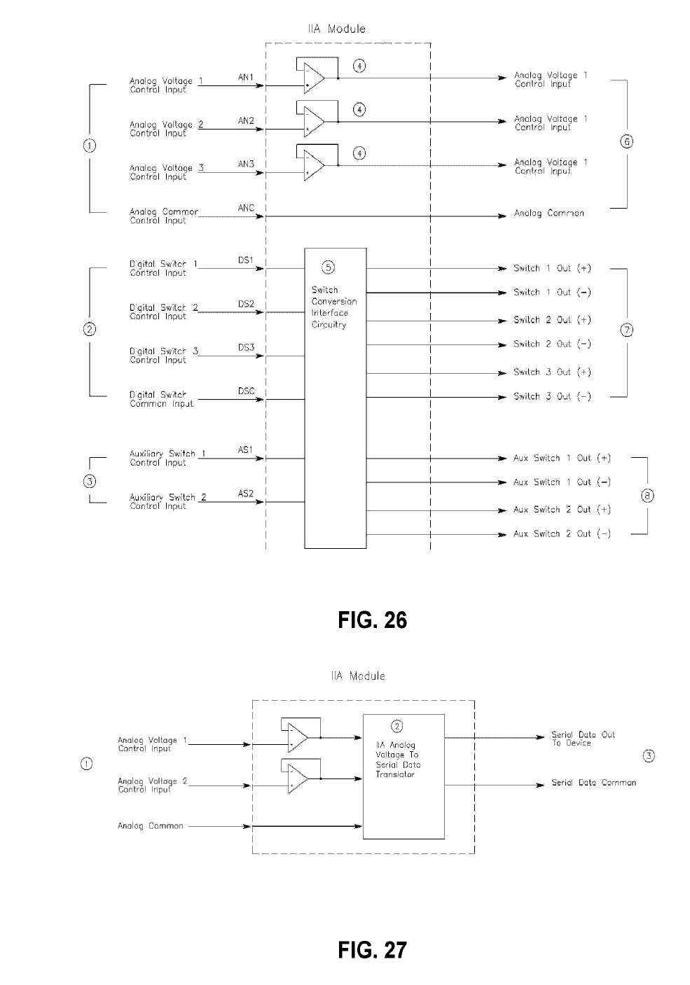

As illustrated in FIG. 26, in an exemplary embodiment of the invention, the conversion circuit may have a plurality of main controls that generate an analog output voltage that may be substantially proportionate to how far the respective foot pedal is depressed. In one aspect, the conversion circuit may have three main controls. The conversion circuitry may provide an analog and a digital switch output to each channel for each main control on the master foot control. As shown in FIG. 26, the conversion circuitry has the ability to control devices that use either on/off switches, analog voltage control, or a combination of the two.

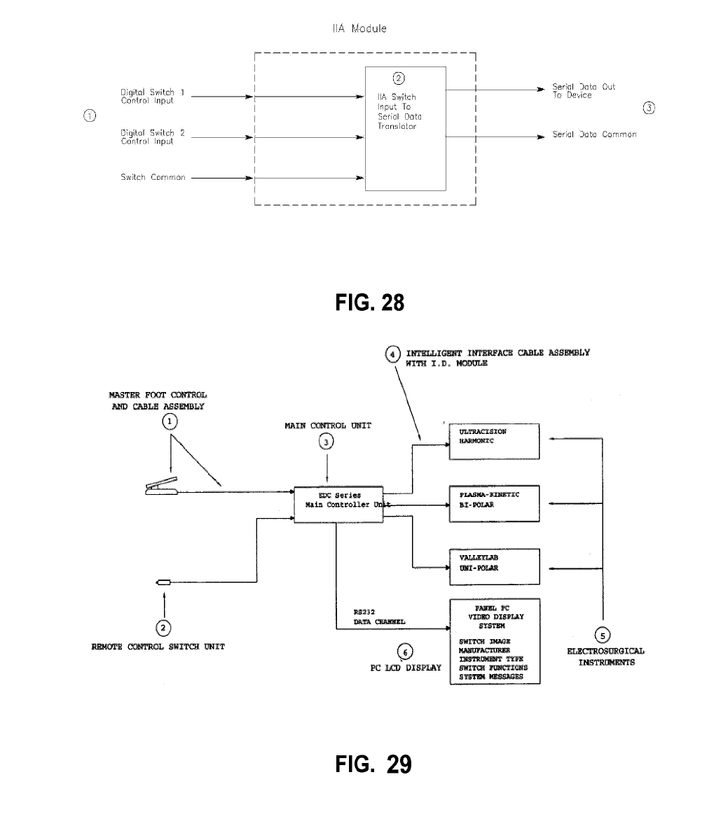

The system may also allow the user to control devices that have serial communication ports for device control with the master foot control. FIGS. 27 and 28 illustrate exemplary embodiments of the invention showing a translator module to translate the signal generated by the foot control switch in either digital or analog format into serial commands for use with non-foot controlled devices. Therefore, devices that are not normally controlled using foot controls may be controlled with the master foot control.

As illustrated in FIG. 5, remote controller 14 functions as a device selector in a manner similar to that in which switch 56 on the operator panel of central controller unit 12 functions as a device selector. In other embodiments of the invention, a device selector can be included, alternatively or in addition, in any other convenient portion of the system. In any embodiment, the device selector is operable by a surgeon or other user to select one of the attached electrosurgical devices for use. In the illustrated embodiment, remote controller 14 includes a suitable housing or enclosure 70 connectable by a suitable length of cable to remote connector 46 (FIG. 2). The exemplary remote controller 14 can, for example, be laid on a suitable surface in the operating room and operated by a nurse in response to instructions spoken by the surgeon during the procedure. Remote controller 14 has elements defining a four-channel user interface: a first channel interface 72 with which two buttons 74 and 76 and a label 78 are associated; a second channel interface 80 with which two buttons and 82 and 84 and a label 86 are associated; a third channel interface 88 with which two buttons 90 and 92 and a label 94 are associated; and a fourth channel interface 96 with which two buttons 98 and 100 and a label 102 are associated. Remote controller 14 also includes a Power LED 104, which, when illuminated, indicates remote controller 14 is powered, and a Remote Online LED 106, which, when illuminated, indicates remote controller 14 is operational. A first channel LED 108 illuminates to indicate that a device has been plugged into channel connector 36 (FIG. 2) and is online, i.e., ready to be selected for use. A second channel LED 110 illuminates to indicate that a device has been plugged into channel connector 38 (FIG. 2) and is online. Similarly, a third channel LED 112 illuminates to indicate that a device has been plugged into channel connector 40 (FIG. 2) and is online, and a fourth channel LED 114 illuminates to indicate that a device has been plugged into channel connector 42 (FIG. 2) and is online.

Remote controller 14 can be operated to not just select one of the electrosurgical devices for use but also, at least in the illustrated embodiment of the invention, at the same time associate each input, e.g., one of the foot pedals, of foot control 16 or other central user control with one of the functions of the selected device. In FIG. 5, the four exemplary devices are: a harmonic device associated with the first channel (and thus with first channel interface 72 of remote controller 14), as indicated by the indicia “Harmonic” of label 78; a unipolar device associated with the second channel (and thus with second channel interface 80), as indicated by the indicia “Unipolar” of label 86; a bipolar device associated with the third channel (and thus with third channel interface 88), as indicated by the indicia “Bipolar” of label 94; and an argon laser device associated with the fourth channel (and thus with fourth channel interface 96), as indicated by the indicia “Argon” of label 102. In this example, the harmonic device has two functions, coagulate and cut, as indicated by the indicia on buttons 74 and 76, respectively. Similarly, the unipolar device has two functions, coagulate and cut, as indicated by the indicia on buttons 82 and 84, respectively. The bipolar device has the same two functions, as indicated by the indicia on buttons 90 and 92, as does the argon device, as indicated by the indicia on buttons 98 and 100. In yet another aspect, the remote controller 14 can be implemented via a PC touch screen.

By pressing the above-described buttons 74, 76, 82, 84, 90, 92, 98 and 100 a nurse or other user can associate each pedal (or other central user input) of foot control 16 (or other central user control) with one of the functions of an electrosurgical device and, by doing so, select the device for use. The button can illuminate in response to it being pressed, or there can otherwise be generated on remote controller 14 or display 18 a suitable indication that it has been pressed. For example, by pressing button 74, which in the illustrated example bears the indicia “COAG ON/OFF,” the nurse or other user can associate the left pedal of foot control 16 (FIG. 1) with the coagulation function that is conventionally associated with the left pedal of the device connected to the first channel. By pressing button 76, which in the illustrated example bears the indicia “CUT ON/OFF,” the nurse or other user can associate the right pedal of foot control 16 (FIG. 1) with the cutting function that is conventionally associated with the right pedal of the device connected to the first channel. As described in further detail below, after the user has made the device selections in this manner, a surgeon depressing the left pedal of foot control 16 results in the electrosurgical device associated with the first channel applying the signals to its electrode in the conventional manner that are intended to coagulate tissue. Depressing the right pedal of foot control 16 results in that device applying the signals to its electrode that are intended to cut tissue. If the user thereafter wishes to select a different electrosurgical device, such as that associated with the third channel, the user can press button 92, which in the illustrated example bears the indicia “COAG ON/OFF,” to associate the left pedal of foot control 16 (FIG. 1) with the coagulation function that is conventionally associated with the left pedal of the device connected to the third channel. In response, button 92 illuminates and button 74 extinguishes to indicate the change. Similarly, the user can press button 90, which in the illustrated example bears the indicia “CUT ON/OFF,” to associate the right pedal of foot control 16 (FIG. 1) with the cutting function that is conventionally associated with the right pedal of the device connected to the third channel. In response, button 90 illuminates and button 76 extinguishes to indicate the change.

Note that the above-described user interface of remote controller 14 allows cross-switching. That is, a user can associate the left pedal (or other central user input) of foot control 16 (or other central user control) with one of the functions of a first electrosurgical device and associate the right pedal (or other central user input) of foot control 16 (or other central user control) with one of the functions of a second electrosurgical device. For example, it may be desired to use one of the electrical surgical devices for cutting and another one of them for coagulation. A user could, for example, press button 82, which in the illustrated example bears the indicia “COAG ON/OFF,” to associate the left pedal of foot control 16 (FIG. 1) with the coagulation function that is conventionally associated with the left pedal of the device connected to the second channel, and press button 98, which in the illustrated example bears the indicia “CUT ON/OFF,” to associate the right pedal of foot control 16 with the cutting function that is conventionally associated with the right pedal of the device connected to the fourth channel. As noted above, the two devices can be similar to each other or can be of different types, have different functions and be from different manufacturers.

Labels 78, 86, 94 and 102 are shown in FIG. 5 as printed on or adhered to enclosure 70, but in other embodiments of the invention (not shown) they can be dynamic, virtual labels on a display, and thus changeable automatically in response to the device type that central controller 12 detects (by reading the intelligent adapter information) has been plugged in to channel connectors 36, 38, 40 and 42 (FIG. 2). In such embodiments, buttons 74, 76, 82, 84, 90, 92, 98 and 100 can also be virtual buttons displayed on a touch-screen display integrated into remote controller 14 that are dynamically labeled in accordance with the functions that central controller 12 detects (by reading the intelligent adapter information) are associated with the two pedals or other device user inputs. Also, as noted above, in other embodiments of the invention, the devices can have functions other than cutting and coagulating, and there can be any suitable number of channels for any corresponding number of devices. Accordingly, the above-described user interface of remote controller 14 would have a corresponding number of buttons or other means for making the associations and other selections described above.

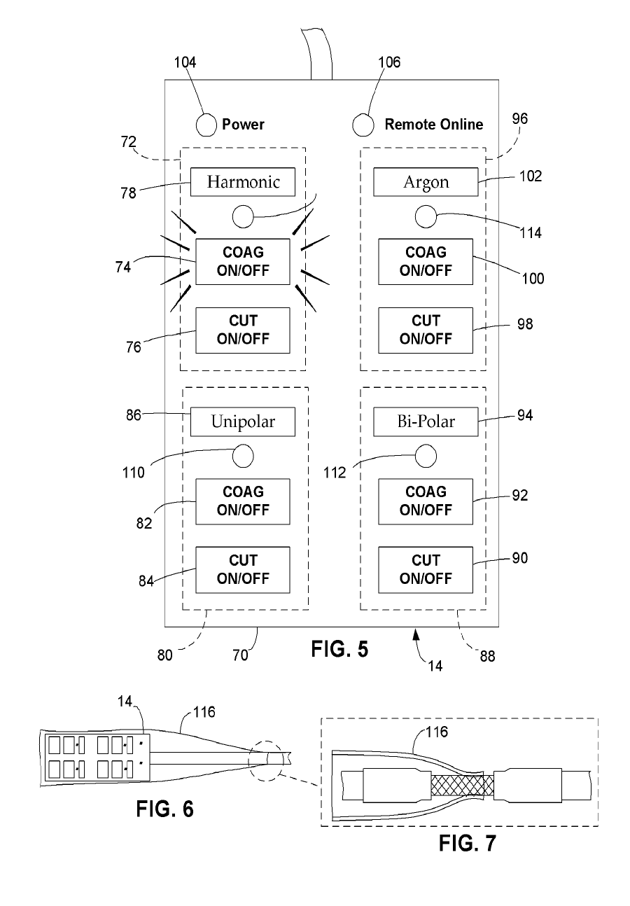

As illustrated in FIGS. 6 and 7, remote controller 14 and a portion of its connecting cable can be covered with a sterile, bag-like, disposable, transparent plastic sheath 116 when used (e.g., by a nurse) within the sterile field of an operating room. Sheath 116 can be made of or coated with a conductive, i.e., anti-static, material and cinched around a portion of the cable at ground potential to bleed static charge to ground, as illustrated in FIG. 7.

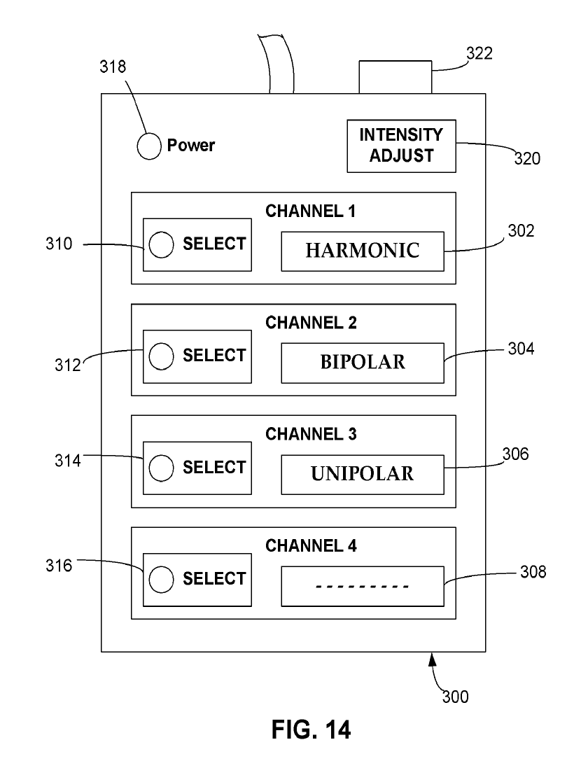

An alternative remote controller 300 is illustrated in FIG. 14. Remote controller 300 is similar to remote controller 14, described above, but in this embodiment it does not have buttons through which an individual pedal can be associated with a device function. Rather, a user can only either select or not select each device. For example, remote controller 300 has four channels, with devices having been connected to the first, second and third channels: a harmonic device associated the first channel and its user interface, as indicated by the indicia “Harmonic” of a label 302; a bipolar device associated with the second channel and its user interface, as indicated by the indicia “Bipolar” of label 304; and a unipolar device associated with the third channel and its user interface as indicated by the indicia “Unipolar” of label 306. No device has been connected to the fourth channel, as indicated by the indicia “ - - - ” of label 308. As with remote controller 14, labels 302, 304, 306 and 308 can be alphanumeric displays that allow the indicia to change dynamically with the type of device that is connected. The first channel user interface has a select button 310, the second channel user interface has a select button 312, the third channel user interface has a select button 314, and the fourth channel user interface has a select button 316. Each button or an LED in the button illuminates when pressed to indicate the selection of the device connected to the corresponding channel. Remote controller 300 further includes an LED 318 to indicate the presence of power, a button 320 through which a user can adjust the intensity of the alphanumeric displays, and a button 322 through which a user can reset remote controller 300 to a default state.

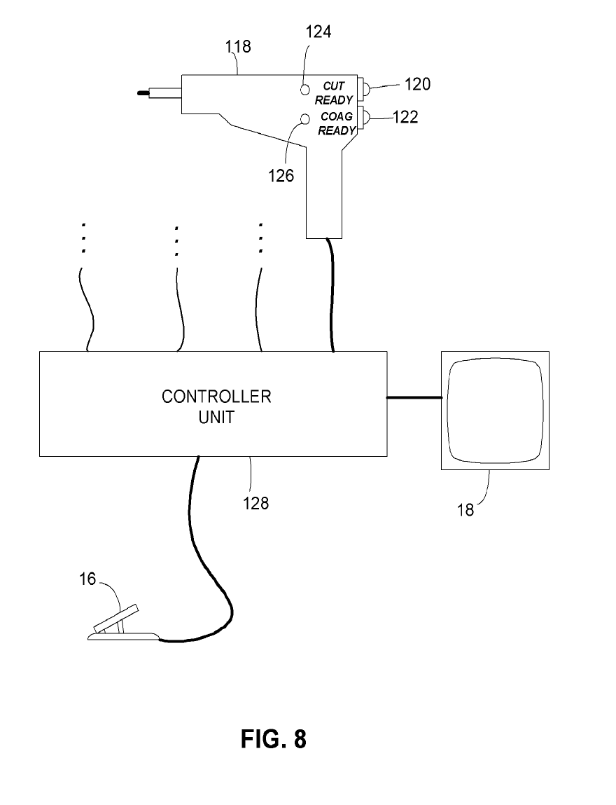

In another embodiment of the invention, illustrated in FIG. 8, an electrosurgical tool 118 itself can include a user interface such as switches 120 and 122 and LEDs 124 and 126, through which a user can select the device for use and associate the pedals of foot control 16 with the functions of tool 118. For example, by pressing switch 120, the user can select and associate the cutting function with the left foot pedal, and by pressing switch 122 the user can select and associate the coagulation function with the right foot pedal. LEDs 124 and 126 illuminate to indicate these selections. Alternatively, in other embodiments, tool 118 can have only one switch, which is used to enable operation of the tool in response to foot control 16. Alternatively, in still other embodiments, switches 120 and 122 can be used instead of foot control 16 to operate tool 118. The central controller unit 128 of such embodiments otherwise is constructed and operates in a manner similar to that described above with regard to FIGS. 1 and 2.

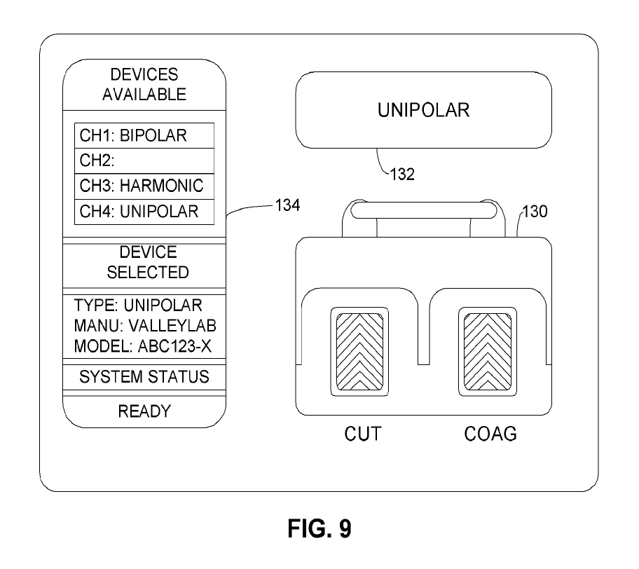

As illustrated in FIGS. 9-11, central controller unit 12 (FIG. 1) can cause information useful to the surgeon or other user to be displayed on display 18 (FIG. 1). The screen shown in FIG. 9 includes a graphical representation 130 of a foot control along with alphanumeric labels “CUT” and “COAG” that indicate, respectively, the left foot pedal is associated with a cutting function, and the right foot pedal is associated with a coagulation function. By viewing such a screen on display 18, the surgeon can quickly and easily ascertain the functions of each pedal without looking away from the surgical field. Note that embodiments of the invention in which the device user control is something other than a foot control, the screen can depict it and its device user inputs, however they may appear. Also note that central controller unit 12 applies the labels to the pedals or other representations of device user inputs in response to the functions of the electrosurgical device that is at that time actually plugged in and selected for use by the surgeon. That is, central control unit 12 applies dynamic labels corresponding to the functions it ascertained by reading the information from the intelligent adapter associated with the selected device.

The screen shown in FIG. 9 further includes an alphanumeric label or indication 132 that the selected electrosurgical device is “UNIPOLAR.” The screen also includes some indications 134 that the devices that have been plugged in (“DEVICES AVAILABLE”) are a “BIPOLAR” device on the first channel (“CH1”), a “HARMONIC” device on the third channel (“CH3”) and a “UNIPOLAR” device on the fourth channel. The absence of an indication adjacent the label “CH2” indicates that no device has been plugged into the second channel. Another indication shows that the “DEVICE SELECTED” is of “TYPE: UNIPOLAR,” is produced by “MAUFACTURER: VALLEYLAB” and is ValleyLab's “MODEL: ABC123-X.” Still another indication shows the “SYSTEM STATUS” as “READY,” indicating that the system is operational and the surgeon can use the selected device.

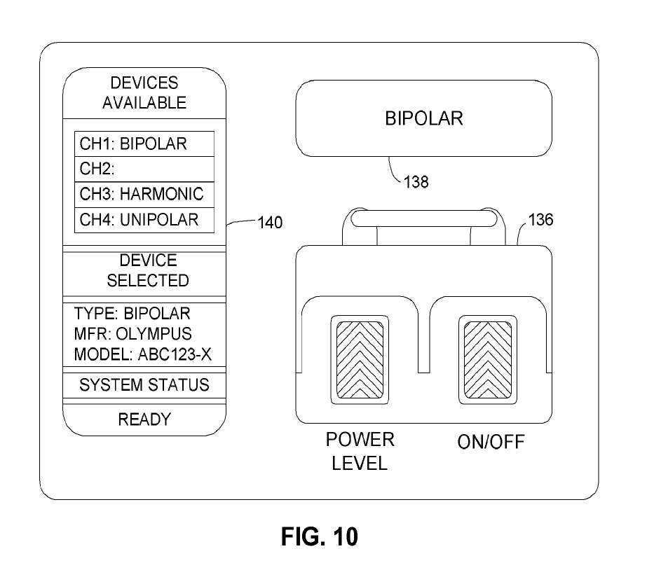

The screen shown in FIG. 10 is similar to that in FIG. 9 and illustrates that, as described above, the displayed information changes as the surgeon selects a different device. The graphical representation 136 indicates that the surgeon has selected a device having, as indicated by the alphanumeric labels, a left foot pedal associated with a “POWER LEVEL” function and a right foot pedal associated with a power “ON/OFF” function. Indication 138 indicates that the selected electrosurgical device is a “BIPOLAR” type. Similarly to FIG. 9, the screen also includes indications 140 that the devices that have been plugged in (“DEVICES AVAILABLE”) are a “BIPOLAR” device on the first channel (“CH1”), a “HARMONIC” device on the third channel (“CH3”) and a “UNIPOLAR” device on the fourth channel. As in FIG. 9, the absence of an indication adjacent the label “CH2” indicates that no device has been plugged into the second channel. Another indication shows that the “DEVICE SELECTED” is of “TYPE: BIPOLAR,” is produced by “MAUF′ACTURER: OLYMPUS” and is Olympus's “MODEL: ABC123-X.” As in FIG. 9, another indication shows the “SYSTEM STATUS” as “READY.” The display may also include at least one verification status indicator and at least one verification command button. In this embodiment, the processor system is programmed or adapted to display an indication of the device verification status of the selected electrosurgical device.

In an embodiment of the invention, the control unit engages a smoke evacuation system upon activation of an electrosurgical device when the function is a surgical function. The smoke evacuation system may remain activated for a predetermined period of time. In an embodiment of the invention the smoke evacuation system comprises a smoke evacuator and an insufflator. As used herein, “surgical function” refers to a cutting or coagulation function of the electrosurgical device. As illustrated in FIG. 10, a bipolar device may have a power level function as well as a surgical function. The control system differentiates between the surgical and non-surgical functions and will activate the smoke evacuation system when the surgeon selects the surgical function. If the surgeon selects the power level function, the control system will not activate the smoke evacuation system. The control system may activate the smoke evacuation system by switch control electrical system or by remote computer command.

In another embodiment of the invention, the control unit can engage a saline flush system upon activation of an electrosurgical device when the function is an arthroscopic function. The saline flush system may remain activated for a predetermined period of time. In an embodiment of the invention the saline flush system comprises a means for pumping a saline solution into the surgical site. As used herein, “arthroscopic function” refers to a minimally invasive operation to repair a damaged joint; whereby the surgeon examines the joint with an arthroscope while making repairs through a small incision. The control system differentiates between the arthroscopic and non-arthroscopic functions and will activate the saline flush system when the surgeon selects the arthroscopic function. The control system may also activate the saline flush system by switch control electrical system or by remote computer command.

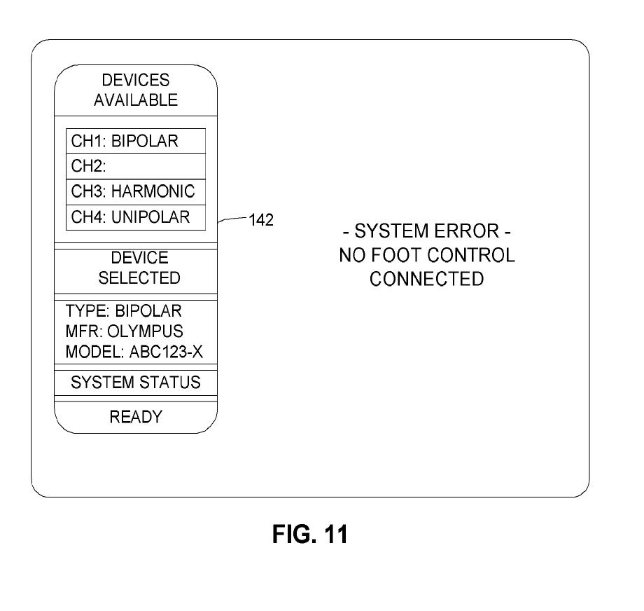

The screen shown in FIG. 11 is similar to those in FIGS. 9 and 10 and illustrates that status information can be displayed. For example, the screen includes a “SYSTEM ERROR” indication, indicating “NO FOOT CONTROL CONNECTED.” As described in further detail below, central controller unit 12 senses when foot control 16 is connected, and if not connected, can display this indication in place of a graphical representation of a foot control to alert the user. Other indications 142 are similar to those described above with regard to FIGS. 9 and 10.

Note that any other status information or other information potentially of interest to a user can be displayed in addition to or alternatively to the information described above, such as an indication that a malfunction or error has occurred (e.g., a failed self-test).

In an embodiment of the invention the processor system is programmed or adapted to record surgical activity, thereby creating recorded information. In an embodiment of the invention, the processor system stores said recorded information.

Central controller unit 12 operates under the control of microcontroller 24, which is programmed to affect the method steps illustrated in FIGS. 12 and 13A-E. It should be noted that the illustrated programming relates to an exemplary embodiment of the invention in which the central user control has a left foot pedal and a right foot pedal as inputs. Nevertheless, persons skilled in the art to which the invention relates will readily be capable of providing programming in other embodiments, in which the central user control is of a type other than a foot control 16 with two such pedals. Also note that in FIGS. 13A-E, the term “CUT” (e.g., “CUT PEDAL,” “CUT SIGNAL,” etc.) is used to refer to the left pedal, and the term “COAG” (e.g., “COAG PEDAL,” “COAG SIGNAL,” etc.) is used to refer to the right pedal. This is done to facilitate understanding by persons skilled in the art, as a large number of conventional electrosurgical devices have a device user control comprising two pedals, in which the function of the two pedals can vary.

When a user first turns on the power, microcontroller 24 performs some initializations and a self-test at step 144. The self-test can include any suitable tests of the type commonly performed to verify proper operation of a microprocessor-based system, such as a CRC check of read-only program memory. If errors are detected at step 146, an error routine is performed at step 148. Although not illustrated in further detail, the error routine can include displaying error indications on display 18 and any other suitable measures such as disabling operation of any connected electrosurgical devices. At step 150, a main control loop routine is entered periodically (e.g., every 6.67 ms in the exemplary embodiment) as a result of a real-time interrupt. As described below, if a user depresses or activates a pedal of foot control 16 at any time during execution of the main control loop, it causes microcontroller 24 to receive a real-time interrupt and act upon the pedal activation by causing a signal applied to the selected device to be adjusted accordingly.

In the main control loop, at step 152, microcontroller 24 checks or senses whether any electrosurgical device has been connected, i.e., plugged in to one of channel connectors 36, 38, 40 and 42 (FIG. 2), since last performing this step. Microcontroller 24 does this by sensing a signal at channel connectors 36, 38, 40 and 42. When this signal is sensed, and if the electrosurgical device associated with that intelligent adapter is not already on-line, microcontroller 24 initiates serial transfer of data from the intelligent adapter memory 68 (FIG. 4) into its SPI subsystem port. If no errors were encountered during the transfer, microcontroller 24 causes the remote controller 14 and display 18 to display the indications described above (e.g., device type, manufacturer, model, etc.) that identify the electrosurgical device on that channel.

At step 154, microcontroller 24 similarly checks or senses at the SPI port whether any electrosurgical device has been disconnected since the step was last performed. If a device has been disconnected during that time, indications that had been displayed are removed or extinguished, or it is otherwise indicated to a user that a device is no longer present on that channel.

Similarly, at step 156, microcontroller 24 senses at its serial communication interface (SCI) subsystem port whether remote controller 14 has been connected, i.e., plugged in to connector 46 (FIG. 2) since the step was last performed. At step 158, microcontrollor 24 senses whether remote controller 14 has been disconnected.

At step 160, microcontroller 24 senses whether a user has pressed switch 56 (FIG. 2). Switch 56 can be a momentary-contact pushbutton or toggle switch that serves as a secondary means for selecting an electrosurgical device, the primary means being remote controller 14. Microcontroller 24 responds to each press of switch 56 y advancing to the next channel. That channel becomes the selected channel, and the previous channel is de-selected. Indications of the selection and de-selection are reflected accordingly in remote controller 14 and display 18.

At step 162, microcontroller processes any messages to be displayed on display 18 in response to the connection, disconnection, selection or de-selection of a device as described above with regard to the main control loop.

If microcontroller 24 receives an interrupt, at step 164, it initializes general software indicators, such as timers, counters and other variables, and determines at step 166 whether there has been a foot pedal activation by reading via I/O circuitry and PPIs 32 and 34 signals received from foot pedal connector 44. At step 168, it verifies that operations are “off,” i.e., that control signals received from connectors 36, 38, 40 and 42 via I/0 circuitry 30 and PPIs 32 and 34 have the expected values and are functioning properly, and returns from the interrupt to the main control loop. If the interrupt was caused by a foot pedal activation, at step 170, microcontroller 24 disables interrupts and, at step 172, performs a routine to process the foot pedal command received at the SPI port, as described in further detail below. Upon returning from the routine, at step 174, microcontroller re-enables interrupts and returns from the interrupt to the main control loop.

The above-mentioned step 172, in which a foot control activation is processed, is illustrated in further detail in FIGS. 13A-E. At step 176, it is determined whether a cutting operation is already in progress. Microcontroller 24 can do this by checking whether a flag or other indicator indicates a state in which a foot pedal associated with a cutting function has already been depressed or activated. If a cutting operation is not already in progress, then at step 178 it is determined whether a coagulation function is already in progress, i.e., the process is in a state in which a foot pedal associated with a coagulation function has already been depressed or activated. If a coagulation function is not already in progress, then at step 180 it is determined whether any system errors are present. Although not specifically described for purposes of clarity, some of the “verify” steps described below with regard to FIGS. 13B-E can include self-tests such as checking RAM 26 and internal memory of microcontroller 24 and checking for proper operation of foot control 16. If any such test indicates an error condition, a flag or indicator is set. Step 180 checks that indicator. If there are system errors, then at step 182 microcontroller 24 causes all signals to the electrosurgical device to be in an “off” state, and returns from the foot control activation processing routine (i.e., returns from step 172).

If at step 180 no system errors were detected, then at step 184 it is determined whether a foot pedal associated with a cutting function has been depressed. If a foot pedal associated with a cutting function has not been depressed, then at step 186 it is determined whether a foot pedal associated with a coagulation function has been pressed. If neither foot pedal has been pressed, microcontroller 24 returns from the foot control activation processing routine.

If at step 176 it is determined that a cutting operation is already in progress, then at step 187 microcontroller 24 verifies that the foot pedal associated with the coagulation function has not been pressed, because such a state could represent a foot control circuit failure or at least an ambiguous condition. If the foot pedal associated with the coagulation function has not been pressed, microcontroller 24 determines at step 188 whether any system errors are present (as described above with regard to step 180). If there are system errors, then at step 190 microcontroller 24 causes all signals to the electrosurgical device relating to the cutting function to be in an “off” or de-energized state, verifies that the signals are off; and returns from the foot control activation processing routine. If there are no system errors, then at step 192 it is determined whether the foot pedal associated with the cutting function is still depressed. If it is not still depressed, then at step 194 microcontroller 24 causes all signals to the electrosurgical device relating to the cutting function to be in an “off” state, sets a master engage signal (“M_ENGAGE”) that enables operation of the system as a whole to “off” or “0”, and returns from the foot control activation processing routine. If that foot pedal is still depressed, then at step 196 microcontroller 24 performs some verifications. These can include: verifying that the master engage signal is asserted (e.g., is “on” or “1”); verifying that a foot command has been detected; verifying that a device that the software indicates is (logically) selected is actually (electrically) selected; verifying that the signals from foot control 16. At step 198, microcontroller 24 determines whether the verifies were successful. If the verifies were successful, microcontroller 24 returns from the foot control activation processing routine. If the verifies were not successful, then at step 200 microcontroller 24 notes that result by setting some system error variables and continues at step 194 as described above.

If at step 178 it is determined that a coagulation operation is already in progress, then at step 201 microcontroller 24 verifies that the foot pedal associated with the cutting function has not been pressed, because such a state could represent a foot control circuit failure or at least an ambiguous condition. If the foot pedal associated with the cutting function has not been pressed, then at step 202 microcontroller 24 determines whether any system errors are present (as described above with regard to steps 180 and 188). If there are system errors, then at step 204 microcontroller 24 causes all signals to the electrosurgical device relating to the cutting function to be in an “off” state, and returns from the foot control activation processing routine. If there are no system errors, then at step 206 it is determined whether the foot pedal associated with the cutting function is still depressed. If it is not still depressed, then at step 208 microcontroller 24 causes all signals to the electrosurgical device relating to the coagulation function to be in an “off” state, sets the master engage signal to “off” or “0”, and returns from the foot control activation processing routine. If that foot pedal is still depressed, then at step 210 microcontroller 24 performs the same verifications as described above with regard to step 196. At step 212, microcontroller 24 determines whether the verifies were successful. If the verifies were successful, microcontroller 24 returns from the foot control activation processing routine. If the verifies were not successful, then at step 214 microcontroller 24 notes that result by setting some system error variables and continues at step 208 as described above.

If at step 184 it is determined that the pedal associated with the cutting function has been depressed, microcontroller 24 disables all interrupts at step 216 and determines at step 217 if the status of the master engage signal is “off” or “0”. If at step 217 it is determined that the master engage signal is off, then at step 218 microcontroller 24 causes all signals to the electrosurgical device relating to the cutting and coagulation functions as well as the master engage signal to be in an “off” state, re-enables the interrupts at step 220, and returns from the foot control activation processing routine. If, however, at step 217 it is determined that the master engage signal is on, then at step 222 it is determined whether the pedal associated with the coagulation function is “off,” i.e., not depressed. If the pedal is not depressed, then at step 224 the select signal state is verified. At step 226, the signal to the device that causes the device to perform the cutting function is asserted or changed to an “on” or “1” state and verified. At step 228, the master engage signal is asserted or changed to an “on” or “1” state and verified.

At step 230, microcontroller 24 determines whether the verifies were successful. If the verifies were successful, microcontroller 24 returns from the foot control activation processing routine. If any of the verifies was not successful, then at step 232 microcontroller 24 disables all signals to the device associated with the cutting and coagulation function as well as the master engage signal and sets system error variables before re-enabling interrupts at step 234 and returning from the foot control activation processing routine. If, however, all verifies were successful, then microcontroller notes that cutting is the active state by setting appropriate variables or flags at step 236, re-enables interrupts at step 238, and returns from the foot control activation processing routine.

If at step 222 it is determined that the pedal associated with the coagulation function is depressed, i.e., not “off,” then at step 240 microcontroller 24 sets an alert indicator that indicates both pedals (cut and coagulation) are “on” or depressed. At step 242 microcontroller 24 then sets all signals to the device that are associated with the cutting function to an “off” state and, at step 244, notes the change in status by setting appropriate variables or flags before continuing with step 234, where it re-enables interrupts before returning from the foot control activation processing routine.

If at step 186 it is determined that the pedal associated with the cutting function has been depressed, microcontroller 24 disables all interrupts at step 246 and determines at step 248 if the status of the master engage signal is “off” or “0”. If at step 248 it is determined that the master engage signal is off, then at step 218 microcontroller 24 causes all signals to the electrosurgical device relating to the cutting and coagulation functions as well as the master engage signal to be in an “off” state, re-enables the interrupts at step 220, and returns from the foot control activation processing routine. If, however, at step 248 it is determined that the master engage signal is “on” or “1”, then at step 254 it is determined whether the pedal associated with the cutting function is “off,” i.e., not depressed. If the pedal is not depressed, then at step 256 the select signal control state is verified. At step 258, the signal to the device that causes the device to perform the cutting function is asserted or changed to an “on” or “1” state and verified. At step 260, the master engage signal is asserted or changed to an “on” or “1” state and verified.

At step 262, microcontroller 24 determines whether the verifies were successful. If the verifies were successful, microcontroller 24 returns from the foot control activation processing routine. If any of the verifies was not successful, then at step 264 microcontroller 24 disables all signals to the device associated with the cutting and coagulation functions as well as the master engage signal, and sets system error variables before re-enabling interrupts at step 266 and returning from the foot control activation processing routine. If, however, all verifies were successful, then microcontroller 24 notes that cutting is the active state by setting appropriate variables or flags at step 268, re-enables interrupts at step 270, and returns from the foot control activation processing routine.

If at step 254 it is determined that the pedal associated with the coagulation function is depressed, i.e., not “off,” then at step 272 microcontroller 24 sets an alert indicator that indicates both pedals (cut and coagulation) are “on” or depressed. At step 274 microcontroller 24 then sets all signals to the device that are associated with the cutting function to an “off” state and, at step 276, notes the change in status by setting appropriate variables or flags before continuing with step 266, where it re-enables interrupts before returning from the foot control activation processing routine.

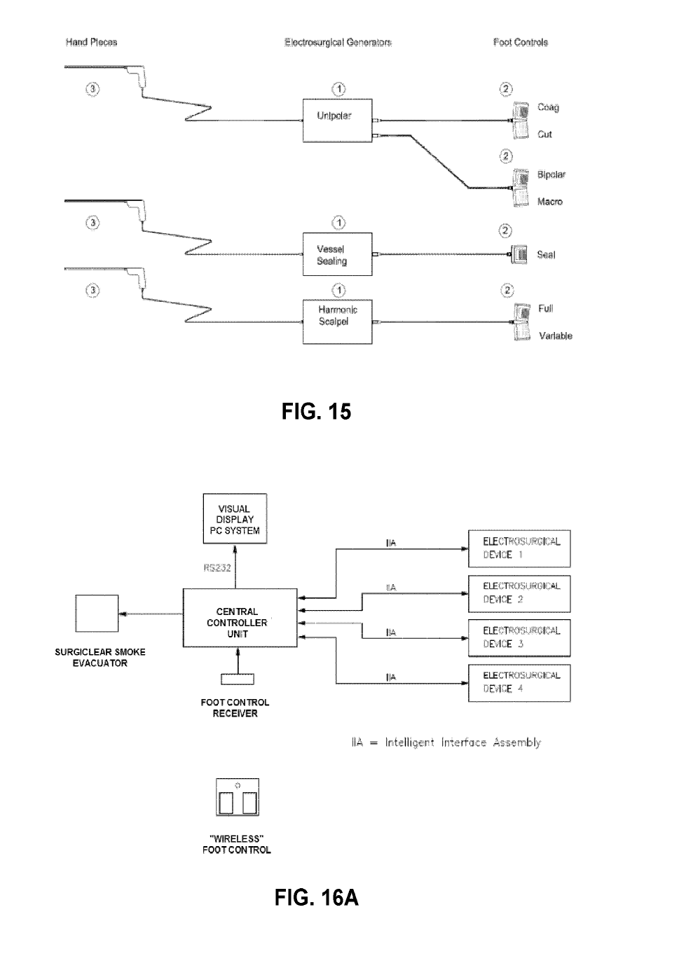

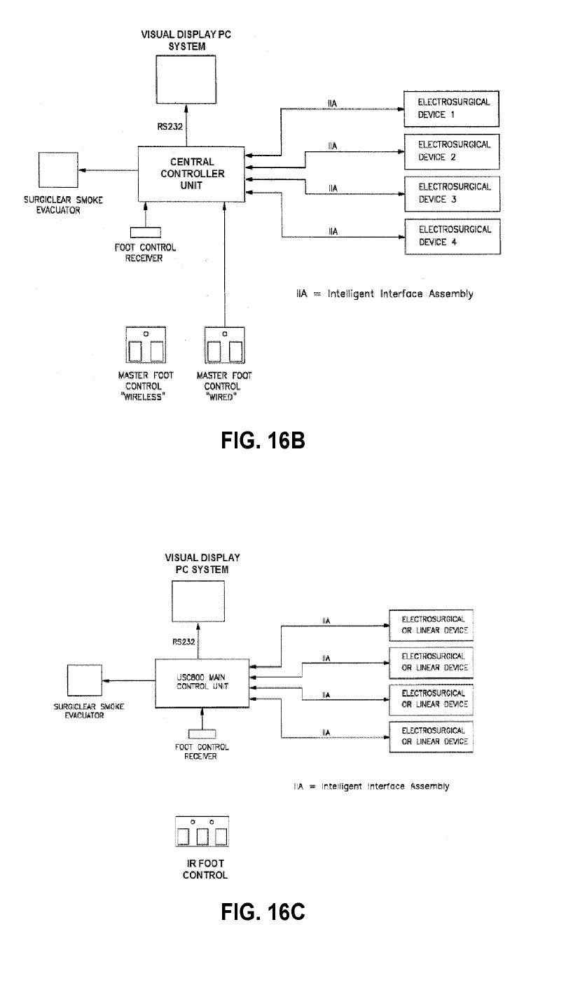

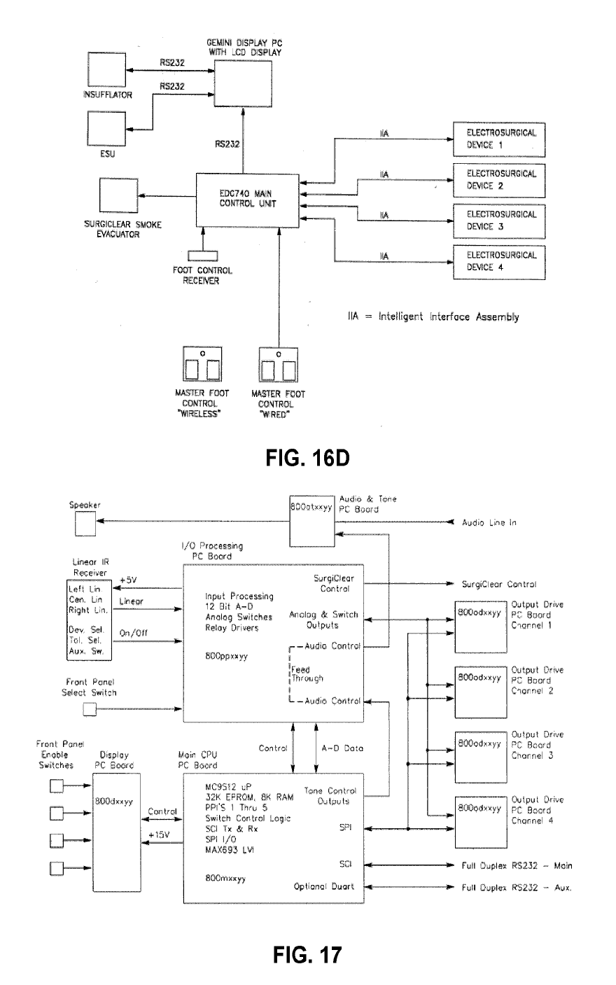

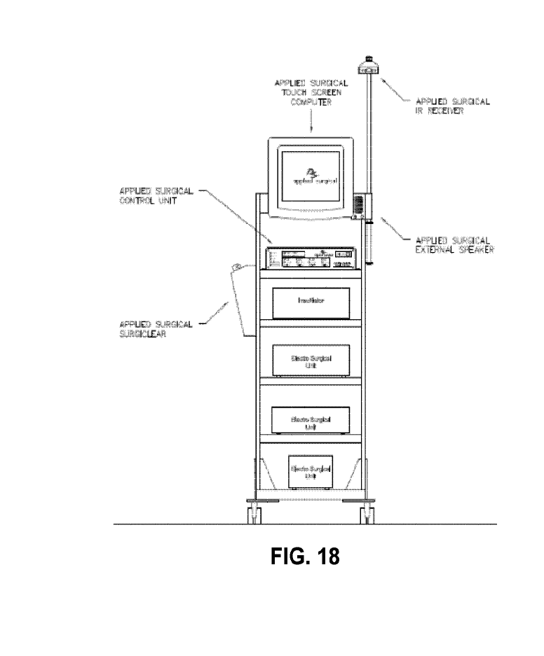

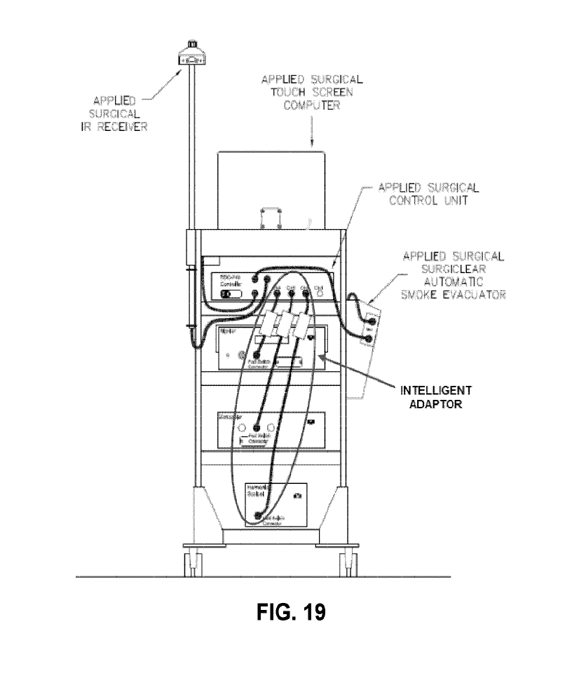

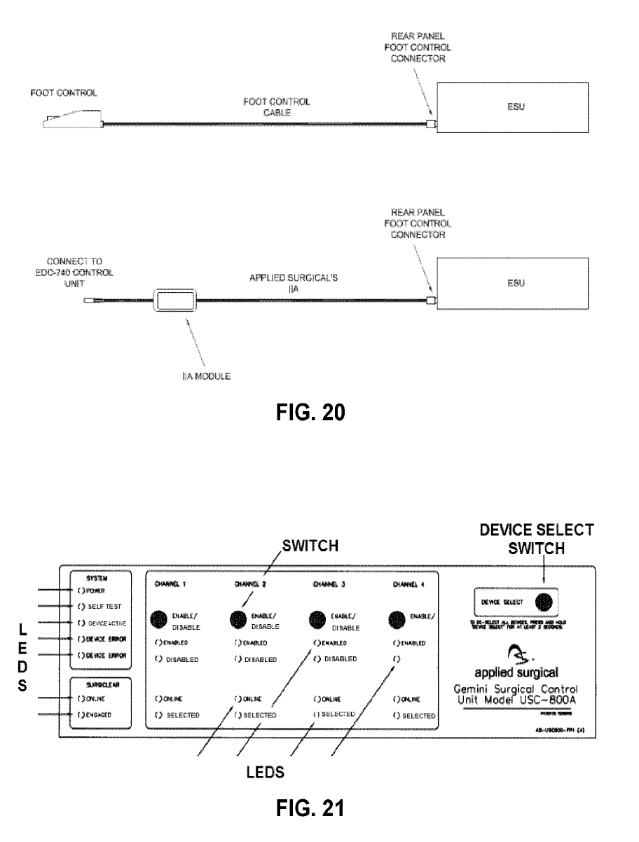

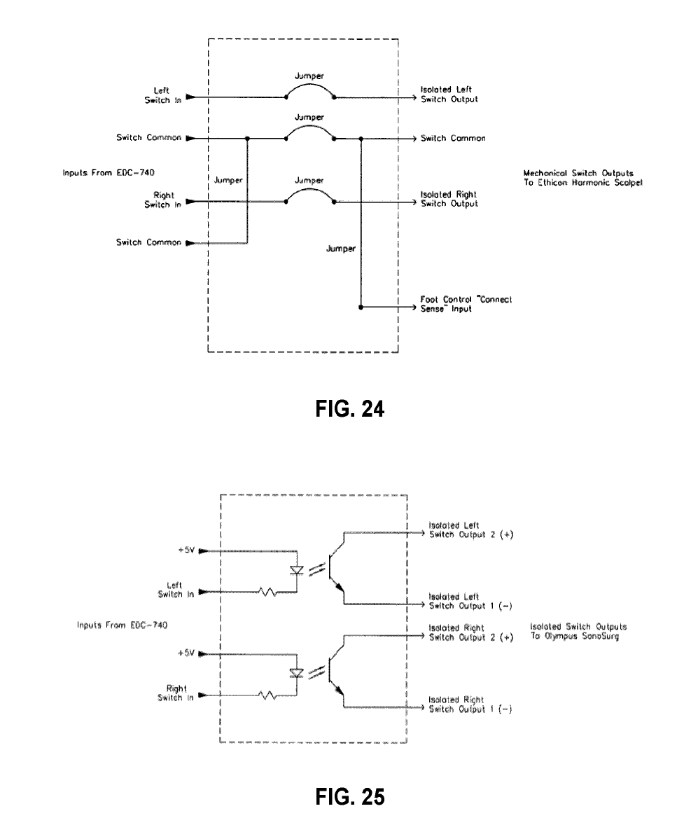

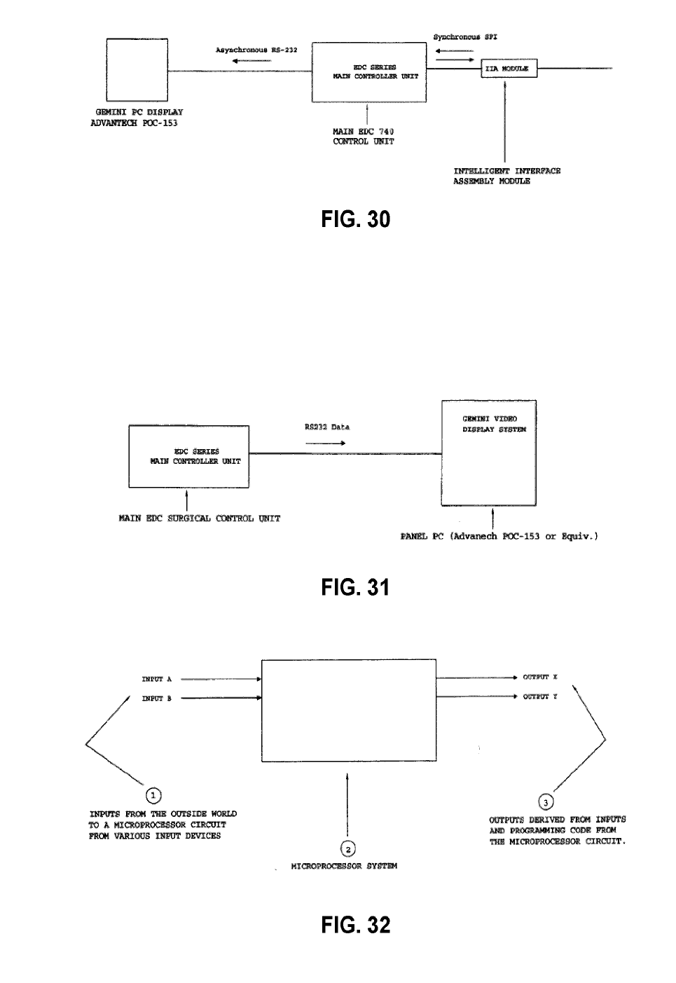

FIG. 15 illustrates conventional connections between conventional ESU's to individual respective foot controls. Referring now to FIGS. 16A-33, in an alternative embodiment of the electrosurgical control system 10, the control system 10 can be configured to combine the operation of both “on/off” controlled electrosurgical devices and “linearly” controlled electrosurgical devices such as, for example and without limitation, shavers and drills. Typical “on/off” controlled electrosurgical and “linearly” controlled surgical devices are manufactured by a wide variety of manufacturers. The control system 10 is configured to accommodate the differing types of electrical interfaces and connectors on each respective manufacturer's foot control assembly. In one aspect, the intelligent adaptor 22 design will be employed to interface the surgical device to the central controller unit 12.

It is contemplated that this embodiment of the electrosurgical control system 10 can optionally: a) provide user friendly user interfaces to the control system 10 that are very simple for the OR staff to operate; b) provide a simple connection modality for any existing or new “linear” or “on/off” activated device; c) provide automatic identification of the connected instrument that can include: a control mode (e.g., linear or on/off); the type of device (i.e., shaver, drill, monopolar, harmonic, and the like); allowed master foot control switches; labeling of the allowed foot control switches; and attributes that describe allowed switch function; d) designate, for on/off control mode devices, the allowed switch state as either on or off (i.e., 1 or 0), and, for linear control mode devices, the allowed switch state as percent depressed (i.e., 10%, 20%, 30%, etc.); e) provide a flexible instrument connection scheme that allows for any instrument type to be connected to any of the channels on the rear panel of the central controller unit 12; f) provide both a physical switch and a communication command method to select a desired surgical device; g) provide for clear and easy selection of the desired surgical device; h) provide for safe and reliable operation; i) provide a foot control 16 having a plurality of linear pedals and a plurality of momentary “on/off” switches; j) provide a linear wireless system, for example and without limitation, IR, RF or the like wireless transmission, for the foot control 16; and k) provide a hand-switch interface for each channel connector that will for example, allow for the integration of the hand-activated instrument into the control system 10.

Exemplary schematic views of this embodiments of control system 10 are shown in FIG. 16A-D. In this exemplary embodiment, the control system 10 comprises the foot control 16, the central controller unit 12, intelligent adaptors 22 and a video display PC system. In one aspect of the control system, the video display PC system acts as a standard user interface that allows for the integration of multiple devices into a single centralized control center. Using the control system as described herein allows surgeons to select from multiple devices and control the various functions of these instruments using a single foot control, which virtually eliminates the potential for accidental device activation and its many, well documented, dangerous consequences and greatly reduces the speed and safety concerns associated with multiple active foot controls on a cluttered operating room floor. As shown, it is contemplated that any surgical device using a foot control with simple “on/off” switches or “linear” control may be connected to any one of the plurality of channel connectors that are typically positioned on the rear panel of the central controller unit (2) via an intelligent adaptor 22.

In one aspect, and as previously discussed herein, the central controller unit 12 is a microprocessor-based control system (i.e., a microcontroller 24) that directs operation of the foot control 16 to one of the connected electrosurgical devices. In one aspect, the intelligent adaptor design allows any existing or future surgical device to be interfaced to the central controller unit 12 by providing an automatic device operation profile. In operation, the selection of a desired surgical device can be performed by either: actuating an integrated device select switch on the foot control 16, or by actuating a device select switch that can be positioned on the front panel of the central controller unit, or by issuance of a communication command to the central controller unit.

In a further aspect, a smoke evacuation unit, such as, for example and not meant to be limiting, the SurgiClear Automatic Smoke Evacuation Unit, can be automatically actuated by the central controller unit 12 whenever a predetermined surgical function is activated. This allows for evacuation of smoke/steam/debris generated by electrosurgical devices during surgical procedures, such as, for example, laparoscopic procedures.

In one general exemplified aspect, general system communication between the central controller unit and the video display PC is via a full-duplex asynchronous RS232 serial data communications. In a further aspect, communications between the central controller unit and the intelligent adaptor module can be via synchronous SPI serial data communications.

In one example, the intelligent adaptor assembly that is used to connect the surgical device to the central controller unit contains a complete device profile for a specific electrosurgical device in its embedded memory. The intelligent adaptor can use a high speed synchronous serial communication channel, such as a Motorola SPI serial communication channel to transfer the data from the intelligent adaptor to the central control unit. In this aspect, the data in the intelligent adaptor is “read only”. That is, serial data is only read from the intelligent adaptor by the central controller unit. In operation, when a surgical device is connected to the rear panel of the central controller unit, a control input line will signal the microprocessor that a device has been connected. The microprocessor will then select the SPI channel for that device and will clock the serial data from the embedded microchip in the intelligent adaptor into the SPI port within the microprocessor. One skilled in the art will appreciate that the intelligent adaptor data can include a complete device profile as required. In another aspect, the central controller unit communicates with the video display PC system via a serial, full-duplex Motorola SCI RS-232 communication channel. These RS232 signals are available at the rear panel “Data Out” connector.

As described above, the purpose of the video display PC system is to serve as the visual and voice guidance aid to the surgeon and operating personnel. This system is a “display only” device and plays no role is actual device control. In various aspects, the display system can indicate the selected device, operation of the foot control with the selected device, system warning messages, and/or system error messages. In one aspect, the video display PC system is an AdvanTech POC-153 medical grade PC with a touch screen LCD display. In one aspect, the video display PC system provides a rich visual environment by indicating, for example and not meant to be limiting, all instruments/devices that are connected, the currently selected instrument/device, operation of the foot control, and/or control system warning and effort messages as applicable.

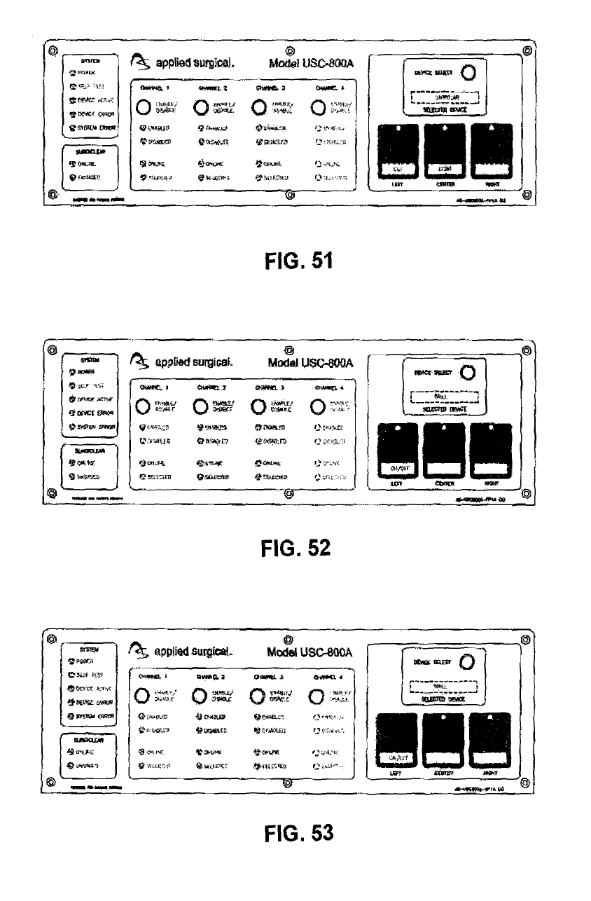

Optionally, and as shown in FIGS. 50-53, it is contemplated that the video display PC system can be replaced with a central controller unit that has integrated visual and voice guidance subsystems. In this aspect, at least a portion of the front panel is configured to display the selected device and operation of the respective controls of the device. In a further aspect, the central controller unit can be configured to provide the voice guidance that was previously allocated to separate portions of the control system. In this aspect, it is contemplated that the central controller unit acts as a stand alone unit that is fully self contained and does not necessarily require the use of the external video display system. Thus, in this aspect, the “integrated” central controller unit provides a rich visual environment by indicating, for example and not meant to be limiting, all instruments/devices that are connected, the currently selected instrument/device, operation of the foot control, and/or control system warning and effort messages as applicable.

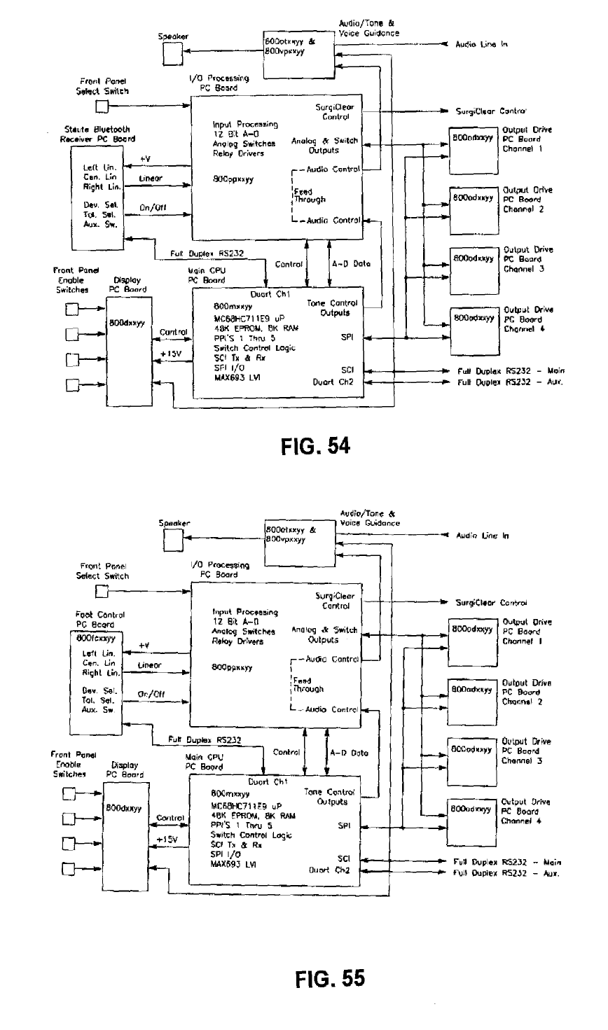

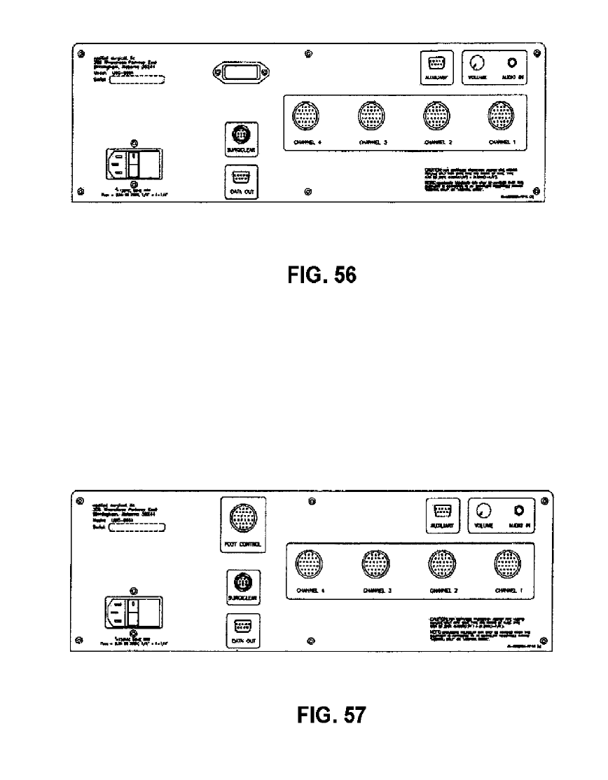

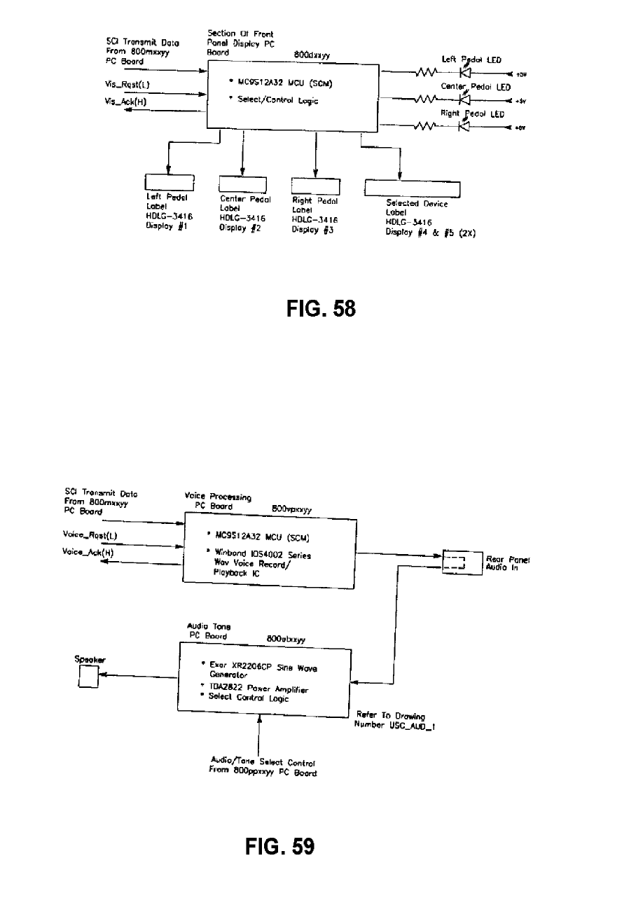

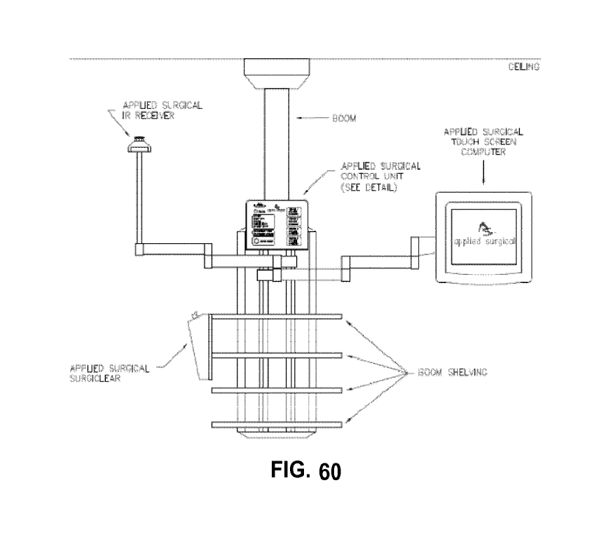

Referring to FIGS. 54-59, it is further contemplated that the control system can be configured to interface with a Bluetooth wireless foot control device using RF transmissions. FIG. 54 shows a general block diagram using the Bluetooth wireless foot control and shows optional aspects including visual and voice processing circuitry. In an alternative aspect, and as shown in FIG. 55 shows a general block diagram using a general foot control with the optional aspects including visual and voice processing circuitry. The options aspects described above are illustrated herein at an exemplary description of a central controller unit that has visual and voice processing integrated therein the central controller unit. FIGS. 56 and 57 show rear panel views of the central controller unit configured for use with the Bluetooth wireless foot control and the general foot control respectively. For use therein the “integrated” central controller unit 12, FIG. 58 shows a block diagram of one exemplary embodiment of the visual guidance circuitry and FIG. 59 shows a block diagram of one exemplary embodiment of the voice guidance circuitry;

The control system 10 can further comprise a means for both linear and on/off mode control. It is contemplated that the control system 10 can be configured to linear type devices (e.g., drills, shavers, and the like) and/or “on/off” electrosurgical type devices (e.g., monopolar, harmonic, and the like). In a further aspect, any combination of these devices may be used simultaneously with the system.

In a further aspect, the control system 10 comprises a means for allowing fast and easy interface to any surgical device. As described above and as shown in FIGS. 23-25, each the intelligent adaptor can comprise an embedded memory with the device profile and a printed circuit interface board. For the control system 10 described in this embodiment, the intelligent adaptor serves as an intelligent extension of the central controller unit and can provide at least one of the following: a) standardization of the interface at the central controller unit's rear panel connectors, which allows for a singular type connector to be employed to connect any existing surgical device and allows for immediate connection to any electrosurgical instrument and does so without requiring cooperative efforts from the device manufacturer; b) automatic identification of the connected device that can include at least one of, a plurality of, or all of: a control mode (linear or on/off), the type of instrument/device (shaver, drill, monopolar, harmonic, etc.), the foot control switches allowed, labeling of allowed foot control switches, and attributes describing allowed switch function; c)identification, for linear mode devices, if the left/center/right on/off switches will be required for operation of the allowed left/center/right switch (“Linear Assist Mode”). In this aspect, the system can also specify activation time as 0=immediate or otherwise as a percentage of switch depression (i.e., 5, 10, 25, 30, etc); d) designating, for on/off control mode devices, the allowed switch state as either on or off (i.e., 1 or 0), and, for linear control mode devices, the allowed switch state as percent depressed (i.e., 10%, 20%, 30%, etc.); e) automatic manufacturer identification (i.e., ValleyLab, Ethicon, etc.) of the device connected to the central controller unit; f) identification, preferably automatically, of the device model number (if applicable) to the central controller unit; g) communication of foot control switch attribute data to the central controller unit to know when to initiate an automatic smoke evacuation cycle; and h) defining functions regarding auxiliary on/off momentary switches, which can include, for example and without limitation, if each switch is allowed, the function label (i.e., Tool Select, Rotation, etc.) for the switch and respective switch attributes.

In various aspects, the intelligent interface assembly described immediately above can thus be used in conjunction with the central controller unit to allow for the connection to virtually any existing surgical device as well as any new products introduced to the market.

In one exemplary aspect, and not intended to limit the selection of a processor system, the microprocessor used in the central controller unit can be a Motorola MC9S12A32 series, which is a member of the Motorola HC12 family series. In this aspect, the MC9S12 family of microprocessors developed by Motorola is high-density complementary metal-oxide semiconductor (CMOS) device, which are advanced and reliable CPU devices with a proven track record.

However, as one skilled in the art will appreciate, normal program flow may be interrupted and, as a result, unpredictable and sometimes uncontrollable system operation is possible. These errors typically are either recoverable or non-recoverable errors. Recoverable errors are normally the result of electrical noise that is induced or radiated and/or from low-voltage brown outs. In a smaller number of cases, programming errors in the code may be the cause. With either problem, design methods are normally employed to eliminate or significantly reduce the chance of these types of errors. The exemplary MC9S12 microprocessor is equipped with self-monitoring circuitry on-chip to protect against system errors. These include, for example and not meant to be limiting, a COP (computer operating properly) watchdog system and a clock monitor fail detection trap. Additionally, an outboard low-voltage supervisory circuit can be added to the central controller unit to further protect the operation of the microprocessor during power-on, power-off and during any voltage brown outs.

Non-recoverable errors are errors that may occur from which there is no way to recover due to permanent damage to the microprocessor device. Permanent damage may occur due to a general and normal device fatality, from damage referred to as ZAP, or from a condition known as SCR latchup. It is contemplated that the control system 10 will employ conventional techniques in electrical circuit design help reduce the possibility of any of these events having an adverse effect on the internal circuits. As one exemplary precaution, the electrical inputs from the various user control inputs can be connected to MAXIM Semiconductor's MAX6817 or MAX6818+−15 KV ESD protected switch debounce IC's. These conventional techniques can help protect the internal circuits from the possibility of ElectroStatic Discharge (ESD) events.

In a further aspect, a microprocessor bypass and override circuit (MBOC) design can be employed in the central controller unit to eliminate any potential problem or adverse effects due to the unlikely event of a microprocessor circuit failure. The operation of this exemplary circuitry is slightly different for devices that employ an on/off control mode (monopolar, bipolar, harmonic, and the like) and those devices that employ a linear control mode (shavers, drill, and like). In the typical operation of a microprocessor circuit that is in total control of the output circuits, the microprocessor receives input data from the input lines “input a” and “input b”. Depending upon the program code located in the microprocessor, the microprocessor makes certain decisions and then generates output information on output lines “output x” and “output y”. Generally, the proper state of the output control lines (output x and output y) from the microprocessor circuit is dependent substantially upon proper operation of the microprocessor circuit. If a device failure occurred that resulted in a non-recoverable situation and in some cases even a recoverable error, the state of the outputs on these lines would be unpredictable, regardless of the state of the input lines (input a and input b).

In a typical operation, assume that “input a” and “input b” is the surgeon's foot control cut and coagulate switch and “output x” and “output y” are the cut and coagulate controls respectively, an electrosurgical handpiece, such as a unipolar hook, has been inserted into the patient, a cut is in progress, and a non-recoverable microprocessor failure occurs where the “output x” enabling the cut operation is now permanently in an enabled or “on” state. In this scenario, the “state” would, unacceptably, continue to exist even if the surgeon released the cut foot switch control.

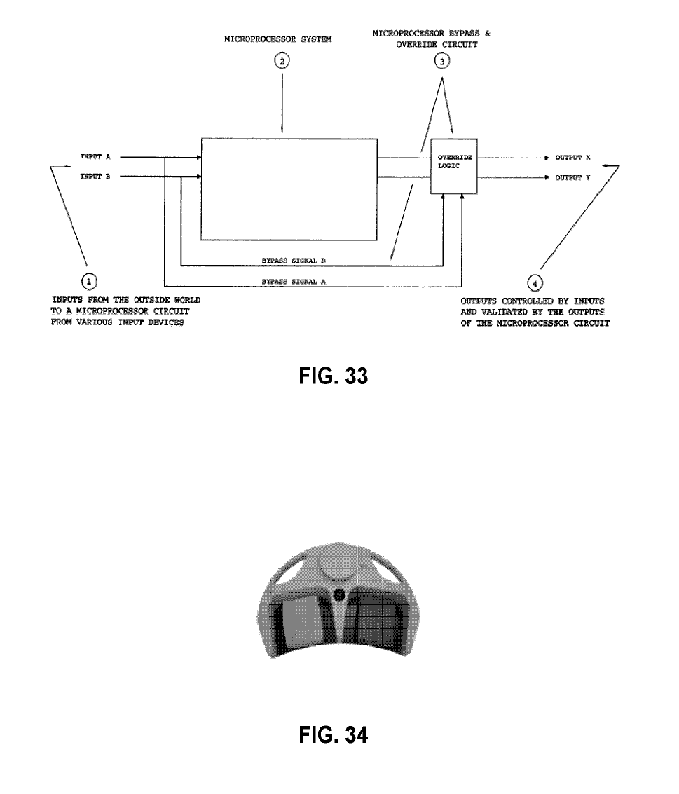

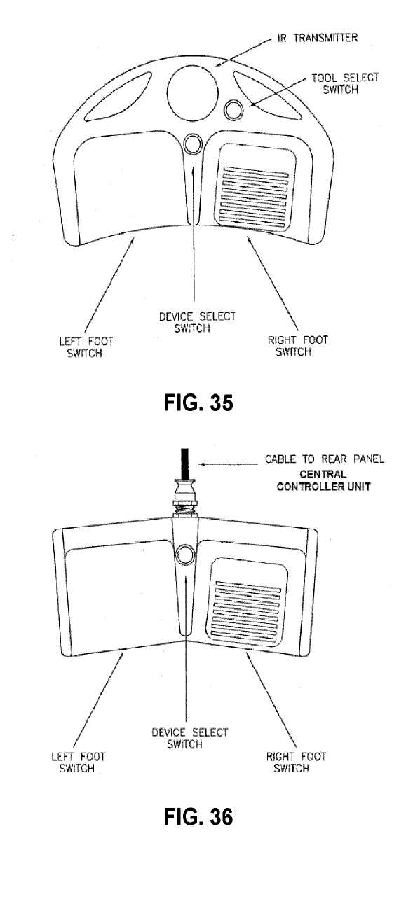

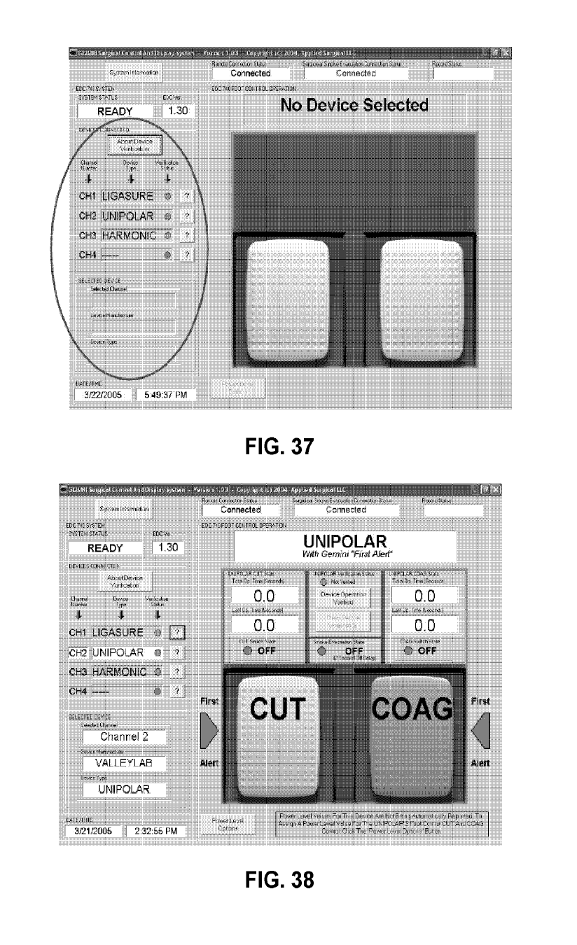

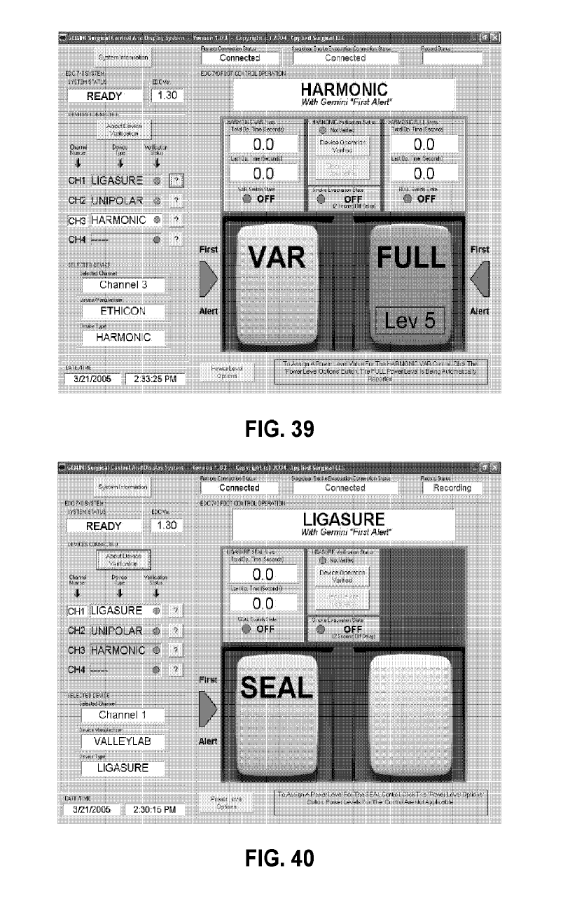

Referring now to the MBOC circuit illustrated in FIG. 33, bypass signals from the left and center foot control switches are routed around the main microprocessor circuit to control the override circuitry in the output left and right switch lines. In this aspect, the left output switch for any device currently in use is controlled by the left switch on the foot control and the center output switch for any device currently in use is controlled by the center switch on the foot control. In one aspect, the microprocessor software can be configured to not initiate a left or center switch operation, only validate it. In this aspect, it is placed under the control of hardware. Therefore, in the operation scenario outlined above, once the surgeon releases the cut switch on the foot control, the override left switch logic will disable the left output switch to the device.

In a further aspect, the control system 10 can comprise a sequential control operation verification (SCOV) methodology/design. In this aspect, the SCOV operates in conjunction with the MBOC circuit as well as providing further control system 10 monitoring to insure proper circuit/software operation. This feature can enhance reliability as well as safety during activation of a surgical device by validating that the microprocessor device select and left/center/right switch control operations are functioning correctly. For example, the control system can be configured such that the verification of proper device selection and the allowance of left, center or right switch operation to continue is broken down into several sequences. In this aspect, the microprocessor can initiate one sequence at a time and verifies that the intended step was correctly performed before continuing to the next step of the validation.

In another aspect of the illustrated embodiment of the control system 10, interface of hand control signals is allowed. These signals enable the system to synchronize the visual/voice guidance and the activation of the SurgiClear automatic smoke evacuator unit with the use of external hand controls. Further, the control system 10 can optionally be configured such that the terminal connector on the “wired” foot control assembly can be connected directly to the terminal connector from an intelligent interface that is connected to an electrosurgical device. This allows connection of the “wired” foot control directly to a specific electrosurgical device. This feature is advantageous in the event the control system 10 is inoperable.

In one exemplary aspect of the system, the central controller unit comprises a four-channel controller. The number of channels is not meant to be limiting, but merely exemplary. In one example, and as shown in FIG. 21, the front panel of the metal housing can be covered with a 0.007″ Marnot mylar overlay that can contain all text and graphical information. In an additional aspect, transparent circular windows can be embedded in this overlay for underlying front panel LED indicators. Optionally, the user interface of the front panel will consist of at least one of the following: LED indicators and/or a momentary push button or toggle SPDT switch for the “Device Select”.

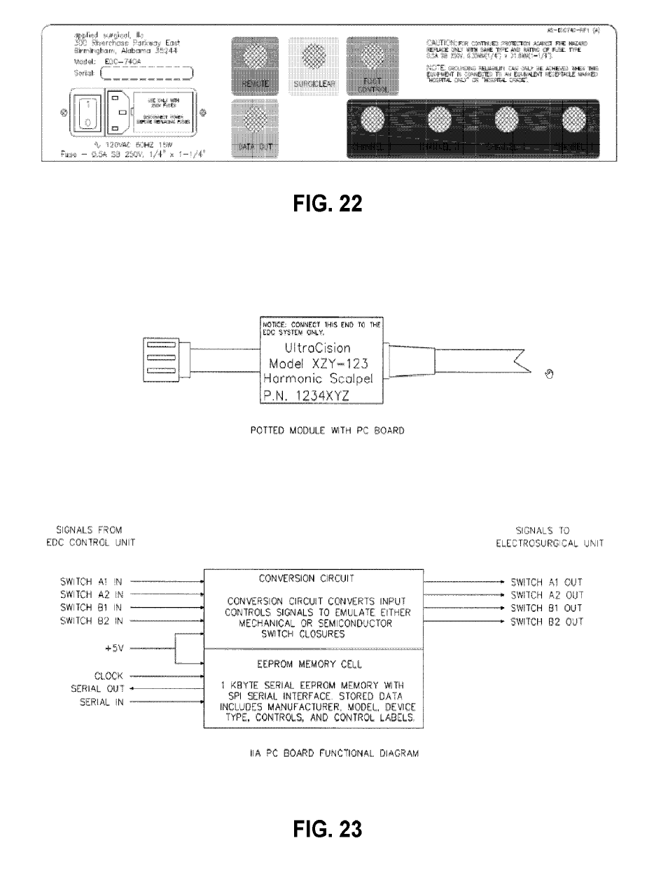

As exemplarily shown in FIG. 22, the rear panel of the central controller unit can exemplarily be covered with a 0.007″ mylar overlay that contains all text and graphical information. Optionally, the user interface of the rear panel will consist of at least one of the following: a medical grade power entry module with switch, fuse and filter, a data out connector, a SurgiClear connector, a master linear foot control receiver interface connector, a plurality of surgical device interface connectors (for example, Channels 1 through 4), an audio In Connector, and a volume control. In one example, LEMO connectors will be used.

In various aspects, it is contemplated that the rear panel connectors on the main controller unit will be selected so that no possibility exists for connecting the wrong item to the wrong connector. Thus, in one aspect, each connector can be unique in pin configuration.

In one aspect, the front panel can further comprise a plurality of momentary push-button switches to either “ENABLE” or “DISABLE” a specific channel. It his aspect, it is also contemplated that associated “ENABLED” or DISABLED” LED indicators would be provided to indicate the status of each connected device. In one aspect, there can be one momentary push-button switch for each device connected to the central controller unit. As exemplarily shown in FIG. 21, four enable/disable switches are provided for a device having four respective channels. In this aspect, the switch allows the user to “Disable” devices when they are not required in the device selection sequence.

Each “ENABLE/DISABLE” switch input circuitry generates a control input signal that will toggle the state between “Enabled” and “Disabled”. In various aspects, the “ENABLE/DISABLE” switches and corresponding LED indicators will function as follows: a) the channel cannot be the “Selected” channel; b) upon a successful device connection, a device will be “Online” and Enabled by default, included in the “Device Select” sequence, and the corresponding “Enabled” LED indicator will be on and the corresponding “Disabled” LED indicator will be off; c) pressing the “ENABLE/DISABLE” switch will toggle to the “Disabled” state and the corresponding “Enabled” LED indicator will be off and the corresponding “Disabled” LED indicator will be blinking; d) pressing the switch again will then toggle to the “Enabled” state and the device is included in the “Device Select” sequence; e) if no device is connected, both the “ENABLED” and “DISABLED” LED Indicators are off; f) if a device is connected and errors were detected, both the “ENABLED” and “DISABLED” LED Indicators are off.

In one aspect, the foot control that is connected to the main controller unit will be either a “wired” or “wireless” linear foot control. Of course, it is also contemplated that a “wired” on/off foot control can be available for direct connection to electrosurgical devices, via the intelligent adaptor, for back-up operation. This wired on/off foot control can be restricted, in one example, for use as a back-up electrosurgical operation. In this aspect, the control may not be connected to the central controller unit.

In one exemplary aspect, the left, center and right linear controls on the foot control 16 will operate both linearly controlled and on/off controlled devices. In this aspect, each type of instrument control (linear or on/off) can implement the microprocessor bypass and override circuitry. In one aspect, the linear input analog voltages of the Left/Center/Right linear controls can be amplified and passed through an active Butterworth filter.

In a further aspect for linearly controlled devices, the analog voltage from the left, center or right linear switch can be routed via analog switches to the appropriate left, center or right output analog drivers for the currently selected channel. In order to implement the MBOC, comparator circuits monitor the analog input level of each linear control switch (left, center and right). Once the input level of a switch control exceeds 5% of the maximum analog level, the respective comparator circuit toggles and then immediately introduces a 20 mv hysteresis. Then, for each switch, a complementary set of control signals are produced from the comparator outputs in order to implement the MBOC to control routing to the appropriate output channel. In another aspect, a 12 bit A-D converter can be employed to provide measurements of each linear switch input, which enables the verification of the analog input level of each linear switch signal.

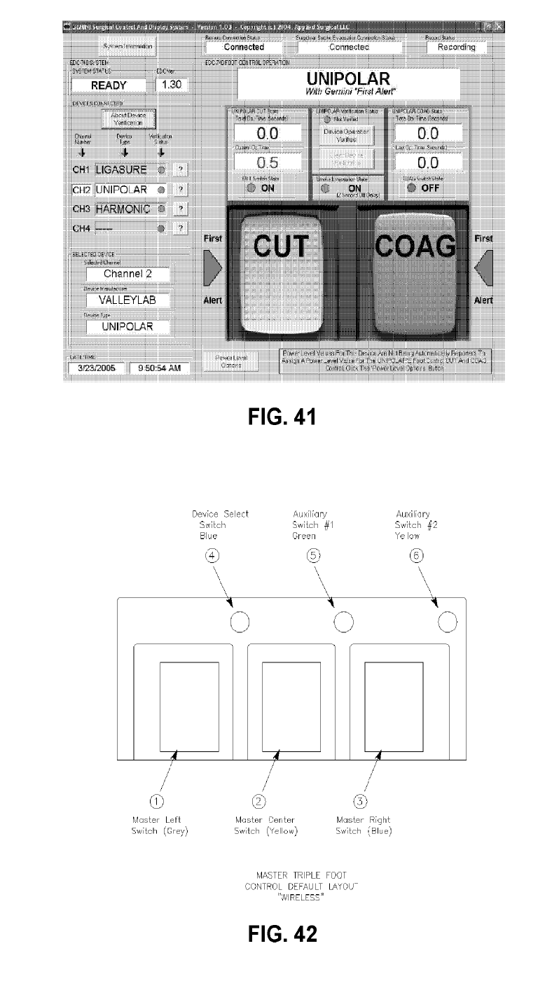

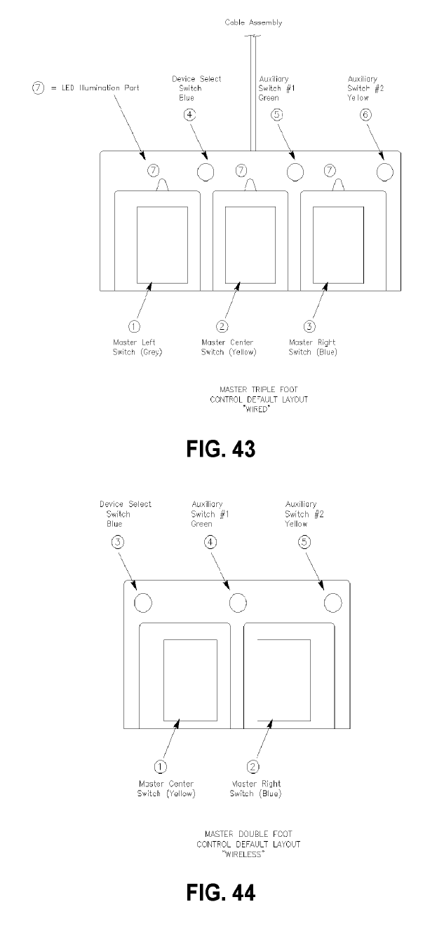

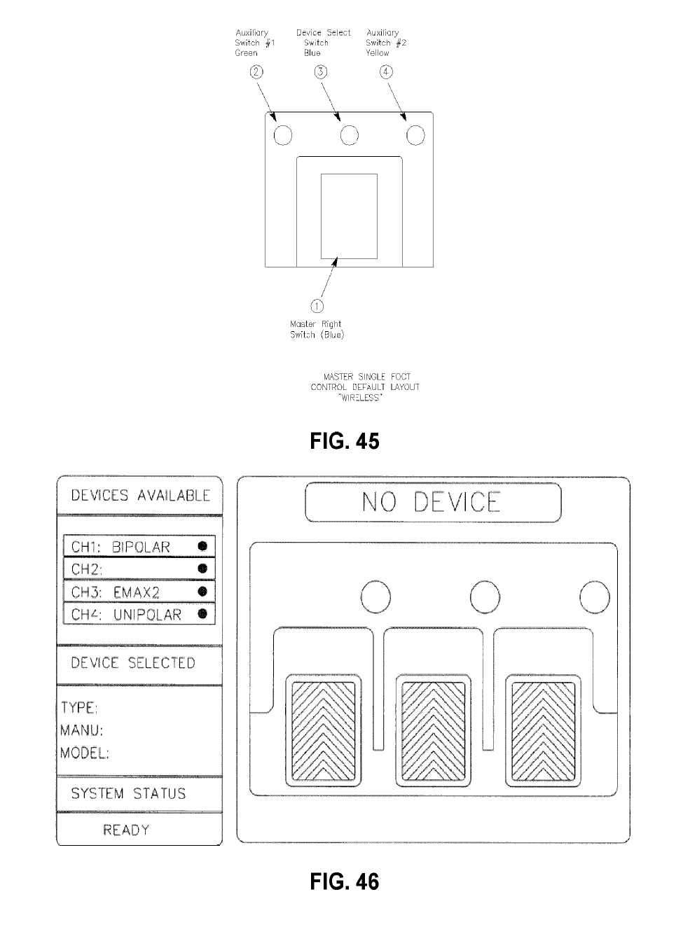

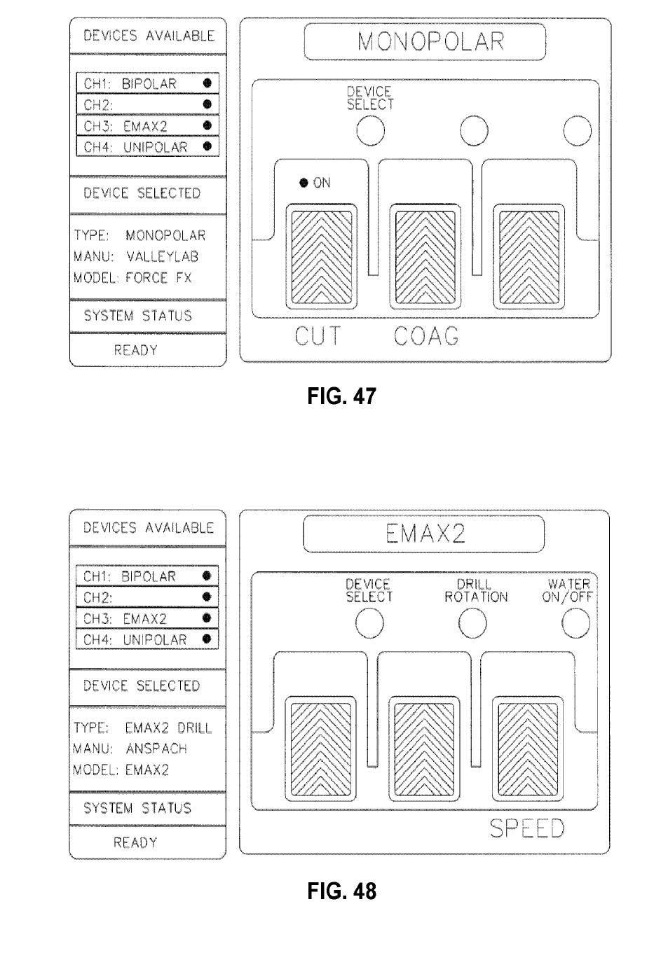

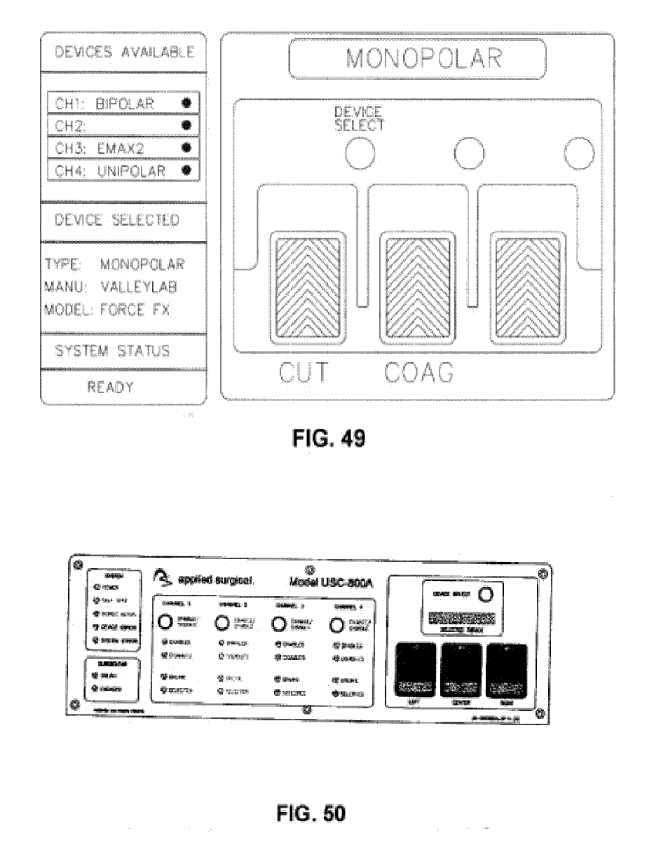

In one exemplary aspect, and referring now to FIGS. 42-49, the foot control 16 can have 3 linear pedals and 3 momentary “on/off” switches. In this example, one of the momentary switches can be dedicated to the integrated device select switch. The other two momentary switches can be used for surgical instrument channel use. In one example, the wireless version of the foot control can be the LineMaster IR three pedal version with three auxiliary on/off momentary control switches. A schematic diagram of the foot control base layout is shown in FIG. 42. In this aspect, the IR foot control receiver can be connected to the rear panel connector that is labeled “FOOT CONTROL”. This exemplary foot control has three linear controls and three auxiliary momentary push-button switches. Further, the IR receiver cable can be terminated with a male LEMO plug connector and can have a feed through signal similar to the intelligent adaptor that can indicate to the central controller unit that the foot control is connected. Additionally, the foot control may be connected and/or disconnected to the central controller unit when power to the central unit is on or off.

In other various aspects, and referring now to FIGS. 42-45, the foot control 16 may have a variety of physical layouts. This will allow the surgeon a preference as to which foot control to use with the main controller. This will prove useful to surgeons requiring differing surgical devices. For example, in general, laparoscopic surgeons use devices that require more controls than devices used by orthopedic surgeons. In this aspect, the foot control will communicate to the main controller as to the exact physical layout of the currently attached foot control 16, this will include the number of linear controls, and the number of on/off or “auxiliary” switches.

In another aspect, the main controller may use Active Control Illumination (“ACI”) to illuminate the active controls used on the foot control for the device currently selected for use. In a further aspect, the illumination may be accomplished by LED ports that may be turned on or off via a communication command from the main controller. As one skilled in the art can appreciate, various illumination techniques may be used. In this aspect, as devices are selected for use, only the controls on the foot control that are active for the particular selected device will be illuminated.

In one alternate aspect, a left, center and right switch press on the foot control can be directed to the currently selected devices. In this aspect, it is contemplated that respective left, center or right foot control functions will be allowed to the selected device only after the system confirms the current configuration and operation. In yet another aspect, the control system 10 can be configured so that illegal switch presses on the foot control (i.e., either a switch not allowed or no device selected) will result in an audible protest beep and follow-up voice guidance. In a further safety feature, the control system 10 can be configured such that, for electrosurgical devices (monopolar, bipolar, harmonic, and the like), the “FirstAlert” feature will provide a short time delay along with audible voice guidance after a switch on the foot control has been pressed until the device is actually activated.

As one skilled in the art will appreciate, surgical devices can be connected to the central controller unit using the intelligent adaptor for each specific device (e.g., Manufacturer & Instrument Type). In one aspect, the terminal end of the intelligent adaptor that connects to a specific electrosurgical device can be marked as such with an ID tag. In this aspect, the terminal end that connects to the surgical device is terminated with a connector that mates with that instrument's foot control connector. In another aspect, the terminal end of the intelligent adaptor that connects to the central controller unit can be marked as such on the mylar decal that is positioned on the potted module and can terminate with a standard LEMO medical grade socket connector (female). This allows for a standard connector that will connect to any of the rear panel connectors on the central controller unit marked CHANNEL 1 through CHANNEL X.

In a further aspect, the control system 10 can be configured such that surgical devices may be connected and/or disconnected to the central controller unit when power to the main unit is on or off. In one aspect, when power is applied to the central controller unit, a feed through signal in the LEMO connector will assert a level to the microprocessor to indicate that a surgical device has been connected. Optionally, this signal is detected as “true” or connected for at least 0.5 seconds before acknowledging the “device connection”. In this aspect, the connection delay acknowledgment allows time for the internal intelligent interface assembly circuitry to power up and stabilize prior to attempting to read the EEPROM, whether internal or not, of the intelligent adaptor.

For electrosurgical devices having on/off control, the terminal connector on the intelligent adaptor assembly can be a male LEMO plug that is configured to mate with a female LEMO socket connector on the wired on/off type foot control assembly. In the event the main unit is inoperable, this foot control assembly can connect directly to the intelligent adaptor assembly.

After the software determines that an device is “connected”, the next step will be to attempt to read the intelligent adaptor assembly EEPROM data for subsequent processing by the microprocessor. If the data read is correct, which can exemplarily comprise start of field indicators, end of field indicators, and/or correct data element frames, then the data is processed as applicable and the device is then considered “Online” and the corresponding “Online” indicator will be set to true for that channel and the corresponding front panel “ONLINE” LED indicator is set to “On”.

If an error is encountered in the intelligent adaptor data read, then a “Device Error” condition is established and the front panel indicators are set accordingly. The “ONLINE” LED indicator will blink and the front panel “DEVICE ERROR” indicator will blink at the same rate. Internal software indicators will be set accordingly. A connected device with an error condition detected may not be selected for use with the system. In one aspect, disconnecting the device can clear the Device Error condition for that device. It is contemplated that, in this case, all other devices that are connected that do not have error conditions will still function.

In an exemplary aspect and not meant to be limiting, the data stored in the internal adaptor assembly can comprise at least one of the following data elements: Device Manufacturer, Device Control Mode, Instrument Type, Device I.D. Number, Left Control Enabled, Center Control Enabled, Right Control Enabled, Left Switch Assist Enabled, Center Switch Assist Enabled, Right Switch Assist Enabled, Left Switch Assist Level, Center Switch Assist Level, Right Switch Assist Level, Auxiliary Switch #1 (Tool Select) Enabled, Auxiliary Switch #2 Enabled, Left Control Label, Center Control Label, Right Control Label, Auxiliary Switch #1 Label, Auxiliary Switch #2 Label, Left Control Attributes, Center Control Attributes, Right Control Attributes, Auxiliary Switch #1 Attributes, Auxiliary Switch #2 Attributes, Left Control Mode Function, Center Control Mode Function, Right Control Mode Function, Left Control FirstAlert Parameters, Center Control FirstAlert Parameters, Right Control FirstAlert Parameters, and the like.

In one aspect, the raw intelligent interface assembly data read from the intelligent adaptor's EEPROM can be verified against a stored intelligent interface assembly EEPROM checksum value. If this checksum matches the computed checksum, the intelligent adaptor data is then parsed and stored into an allocated intelligent adaptor assembly RAM data block. A new checksum is then computed for this data block and stored at the end of the block for future verification. Along with the intelligent interface assembly data, the intelligent adaptor assembly can be configured to provide a hardware input “Fault” indicator signal.

As noted above, in one aspect the system can be configured so that a device must be considered “Selected” before that device can be used with the Master Foot Control. A connected device may be “Selected” by several methods, which include, for example and not meant to be limiting, via the front panel device select switch, the integral foot control device select switch, and/or a transmitted communication to the control system. In operation, if either the device select switches are selected, the device select sequence can occur in numerical order from Channel 1 through Channel X for each depression of the switch. For example, if devices are connected and “Online”, without errors, to all channels, the select sequence will be 1, 2, 3, 4, . . . , X. and then starting back at Channel 1 on the next press. In a further aspect, channels that have no connected device or a connected device with errors will be skipped. When using a communication command, part of the command data will be the desired channel. Therefore, when using this method, the desired channel may be directly selected without passing through unwanted channels. This will save time when multiple devices are employed. Of course, the channel specified must have a connected device recognized as “Online” without any errors to be selected. One skilled in the art will appreciate that once an device is selected, the internal activation control sequence for activating a function on a linear mode device is followed.

As shown in the figures, the front panel of the main controller unit can have multiple indicators. In one example a “POWER” LED Indicator can be provided that is on (continuous, non-blinking) when AC power is applied to the unit. A “SELF TEST” LED Indicator can indicate that the central controller unit is undergoing a complete self test procedure and is switched on (continuous, non-blinking) only during the power-on self test procedure. Further, a “SURGICLEAR ONLINE” LED Indicator indicates that the SurgiClear system is connected with the power on. If the SurgiClear system is either not connected or is connected but the power is off, this indicator will not be illuminated. Also, a “SURGICLEAR ENGAGED” LED Indicator indicates that a SurgiClear Automatic Smoke Evacuation cycle is in progress. In one example, the control line that enables the SurgiClear device can be activated whenever an electrosurgical device is energized and will have a off delay, for example, a five second delay, after the surgical device is de-activated.

In another example, an “ONLINE” LED Indicator indicates that a device connected to that respective channel has been recognized by the control system and is capable of being “selected” for subsequent use. As one would appreciate from the explanation above, the use of the intelligent adaptor for a specific electrosurgical device allows that electrosurgical device to be connected to any one of the channel connectors on the rear panel of the unit. When an electrosurgical device is connected to a rear panel connector, a feed through signal in the connector indicates to the microprocessor that an device is connected. This signal, in each of the panel connectors, can be sampled each time through the main control loop.

In operation, and as discussed above, when this feed through signal is sensed, and if the device is not already online, the microprocessor begins the serial transfer of the data frame from the intelligent adaptor into the SPI port of the microprocessor. The microprocessor then checks the integrity of the data frame by performing a checksum calculation and comparing that with a stored checksum value. If no errors exist, the “ONLINE” LED indicator for that channel is switched on (continuous, non-blinking). The device is then, logically speaking, “online”. If an error is discovered in the checksum value or data format, the “ONLINE” LED indicator is switched on with a blink rate of approximately 2 Hz. Also a “DEVICE ERROR” LED indicator is switched on with the same 2 Hz rate. All other devices without errors will continue to operate normally. When the device with the indicated error is removed by disconnecting it from the rear panel, the device error condition will self correct.

In a further aspect, because the feed through signal in each rear panel device connector is scanned each time through the main control loop, when a device is disconnected that was previously “online”, that device is immediately removed, logically speaking, from the system. Thus, the “ONLINE” LED indicator is switched off. If the device was in a “Selected” state, the “SELECTED” LED indicator will also be switch off.

It is also contemplated that each of the channels in the system can have a “SELECTED” LED indicator that can be, in one example, located directly below the “ONLINE” LED indicator. The “SELECTED” LED indicates that a specific device is ready to perform a foot control operation as received from the foot control 16. In one aspect of the system only one device may be selected at a time and the device must be “online” before it can be selected. When an electrosurgical device is selected, the “SELECTED” LED will be switched on (continuous, non-blinking). In a further aspect, an “ACTIVE” LED can indicate that a left, center or right foot command operation is in progress. When a legal foot control switch is sensed, this LED will be switch on (continuous, non-blinking). If a foot control switch is pressed illegally (that is the switch is not allowed for use) an illegal audible protest beep will occur.

Optionally, a “DEVICE ERROR” LED can be provided that indicates that the central controller unit has detected a device error condition. In one example, the “DEVICE ERROR” LED indicator can be configured to blink at approximately a 2 Hz rate when a device error is detected by the system microprocessor. This LED is used in conjunction with the “ONLINE” LED indicators to communicate to the user which device has the error. As noted above, device errors will self correct when the identified defective device is disconnected from the central controller unit and will not interfere with the operation of other devices which do not have errors detected.

In another aspect, the control system 10 can comprise a “SYSTEM ERROR” LED indicator. In operation, it is possible for system errors to be reported during the power-on and self test procedure. During execution of the main control program, error detection software can be configured to be active and running. If an error is detected internal to the system during operation, the “SYSTEM ERROR” LED will be switched on and further system operation will halt. A specific failure message will be displayed in the remote unit's displays. Optionally, an audible alert will also be turned on.

In a further aspect, to aid in control system set-up and proper system configuration, the video display PC system can be used to provide the user with complete information regarding at least one of the following: the status of the central controller unit, the status of the remote control unit connection, the status of the foot control connection, and/or the status of the SurgiClear connection status. In yet another aspect, to aid in diagnosing system warning or error conditions, the video display PC system can be used to provide the user with complete information regarding the cause and possible solution to at least one of the following: errors or warnings in the central controller unit, errors or warnings in the device select switch circuit, errors or warnings in the foot control switch circuits, and/or information regarding the status, errors or warnings with a connected surgical device.

In an additional safety aspect of the system, the microprocessor bypass and override circuit eliminates the possibility of the microprocessor software activating an output switch erroneously. In this aspect, the microprocessor plays a supervisory role and can disable all output switches but does not have authority to originate the activation of an output switch. This hardware circuit eliminates or minimizes the following software related failure possibilities: a) the left/center/right switch is pressed on the foot control and a failure in the software or input sensing results in the opposite switch being activated; b) a left/center/right switch has been correctly activated and a subsequent failure in the software or input sensing circuit resulting in the switch being stuck in the activated mode; and/or c) a failure in the microprocessor software results in the activation of a left/center/right output switch when no switch has been pressed on the foot control.

In this aspect, the microprocessor controls which device is currently selected for use. Therefore the design is configured to provide a means for the system to verify the instrument selection circuitry. In one aspect, the function of the circuitry to control and verify correct instrument selection is a component of the sequential electrosurgical operation verification (SEOV) circuit. Here, once a switch is pressed on the master foot control, the microprocessor, through the SCOV circuitry, verifies that the system is functioning properly and if so, allows the switch operation to continue to the selected device. The microprocessor can be configured to continuously monitor all circuit operation (via the SCOV circuitry) and if a problem is detected, it can override device activation and prohibit any devices from being activated even though the left/center/right switch on the foot control is being pressed.

In operation, when devices are recognized by the central controller unit and are displayed on the visual display PC system, the surgeon can, at any time, use the foot pedal to select any of the available devices. Using the device select button on the foot pedal, the surgeon may at any time use the foot pedal to select any of the available devices. Using the device select button on the foot pedal, the surgeon may toggle between each of the connected instruments. In various aspects, the operating personnel can be alerted to the device that is selected by a visual screen display and/or by a voice notification.

It will be appreciated by those skilled in the art, these rich visual and voice notifications and the fact that only one device can be selected and fired at once, combined with the elimination of other footswitches on the operating room floor, have significant safety and efficiency implications. In one aspect, the control system 10 make accidental device activation virtually impossible, which thereby greatly reduces electrosurgical burns (whether internal or external), eliminates the primary means of ignition of surgical fires, and significantly eases the mental burden placed on the surgeon to maintain constant awareness of device selection and left and right foot pedal assignment. In addition to these benefits, the nurses and support staff no longer have to attend to the surgeon's “foot pedal dance”. This benefit greatly reduces OR staff fatigue, saves valuable time each time the surgeon changes instruments or requires verification of instrument selection, and allows them to direct their focus toward the patient and not at the surgeon's feet. Finally, the elimination of a plurality of cords and cables and up to 3 additional foot pedals makes for a much safer environment for circulating nurses and much more flexibility and freedom of movement around the operating table for the surgeon, which has both ergonomic and efficiency implications.

When a surgeon selects an electrosurgical device to use, several things can occur on the video display PC system monitor. As mentioned, a voice notification alerts the surgeon to the device that he or she has selected, and the screen visually displays both the instrument type (i.e., monopolar, bipolar, harmonic, and the like) and the device manufacturer. In addition, the monitor displays simulated left and right foot pedals as they actually appear on the foot control with the functionality that corresponds to each foot pedal overlaid over those simulated foot pedals, as seen in representative FIGS. 38 (unipolar), 39 (bipolar), and 40 (ligasure).

When the surgeon fires the instrument/device by depressing a respective foot pedal of the foot control, the screen provides voice notification by communicating the function (i.e., ‘CUT’, ‘COAG’, ‘SEAL’, etc.) that corresponds to the depressed foot pedal and further visual verification by simulating the depressing of that same foot pedal. Substantially simultaneously, the central controller unit communicates to the display monitor data related to the activation of that device such as current operating time (the running duration of time that this device function has been continuously activated during this firing) and total operating time (the total amount of time that this device function has been activated during this surgery). As exemplarily shown in FIG. 38, in the case of a two-function device (i.e., ‘CUT’ and ‘COAG’), this data is displayed for each function. As desired, the data can be recorded for instructional and legal purposes.

In another aspect and as described above, the integration of the various independent electrosurgical instruments to the central controller unit allows for the determination of when and for how long evacuation of smoke is required for the various instruments in use. In one aspect, based on the smoke-producing characteristics inherent to each energy modality (and each foot pedal function within that modality), the central controller unit is programmed to send commands to the smoke evacuator, which automatically operates in accordance with these commands. This provides an automatic, closed-loop smoke evacuation system that can yield tremendous benefits to the patient and the surgical team as the field of view remains clear throughout the surgery. This allows the surgeon to no longer have to suspend the surgery to vent out the abdominal cavity via an opened trocar or cannula. As a result, the risk of injury to the patient due to misapplication of the tip of the electrosurgical devices resulting from an unclear field of vision is minimized and the risk of patient harm due to the hazardous accumulation of CO in the intra-abdominal cavity and the resultant elevations of carboxyhemoglobin (COHb) is minimized. Further, the numerous documented risks associated with repeated inhalation of toxins from surgical smoke by surgeons and OR staff are reduced. FIG. 41 illustrates the smoke evacuation system turned on during a “CUT” operation.

In addition to the features mentioned above, the control system 10 can also allow for two wireless foot controls to be used, one on each side of the table, either of which can at any time activate any of the electrosurgical instrument employed during the surgery. This functionality can be a tremendous time saver in more complicated surgeries that required more than one surgeon.

In another aspect, the control system 10 can be configured to implement a brief delay between foot pedal depression and actual device activation. This delay can allow surgeons to verify the selected instrument and function selection both audibly and visually before the device is actually fired. This “grace period” is yet another safety check in the control system with regards to the prevention of accidental device activation. It is contemplated that this feature can be disabled as surgeon preference dictates.

In yet another aspect of the control system, the parameter of a conventional external insufflator may be monitored via an RS232 port on visual display PC system and/or may be communicated to the central controller unit. In one aspect, this is a monitor-only feature and does not actively control any operating mode of the external insufflator. In various aspects, the monitored parameters can include: preset pressure, actual pressure, and insufflator warnings and errors. It is of course contemplated that, in an alternative aspect, that the central controller unit could communicate operational signals is response to the monitored parameters to control the operation of the external insufflator. In a further aspect, the control system 10 can allow the operator to specify a “differential pressure” between preset and the actual pressures. In this aspect, if the actual pressure is below this pressure differential, the microprocessor will issue command signals to inhibit activation of the SurgiClear smoke evacuator. Optionally, the control system 10 can detect any warnings or errors reported by the insufflator to indicate to the user when this feature is operational.

In another aspect, the control system 10 can be configured to allow for the monitoring of electrosurgical handpiece selection. As one skilled in the art will appreciate, a few instruments allow operators to select from two or more connected handpieces. Typically, for this type of instrument that has a conventional RS232 communication port, the “selected handpiece” may be determined via the RS232 communication port. Monitoring this information allows the selected handpiece to be integrated into the visual and voice guidance features of the system 10. In one example, the communication ports on the visual display PC system can be used to implement this function. In one aspect, this is a monitor-only feature and does not actively control any of the device's operating parameters. Alternatively, it is contemplated that the system can be configured to incorporate an interface that integrates the operation of the hand controls into the control system 10. This allows the benefits of the control system to be extended to hand controlled instruments.

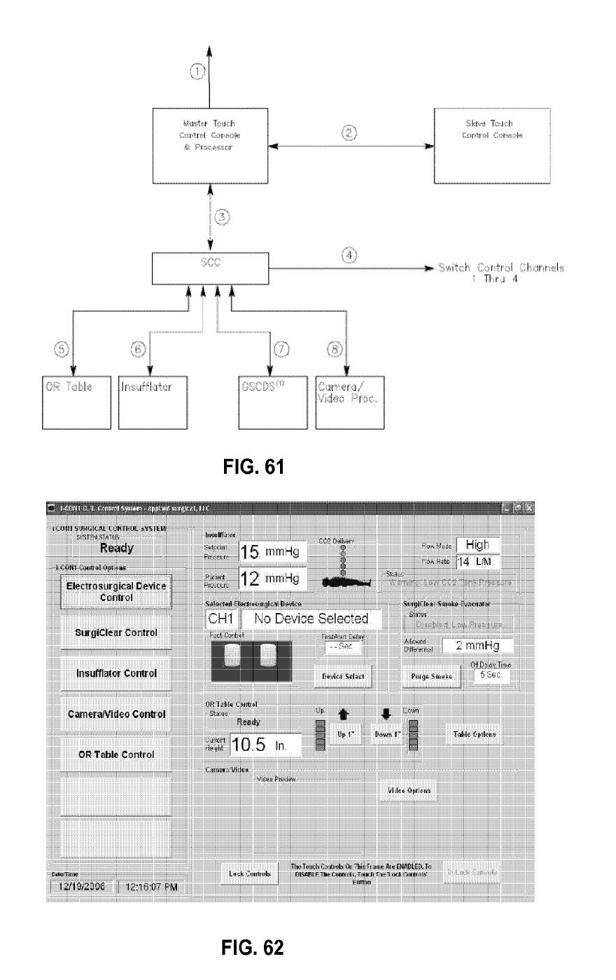

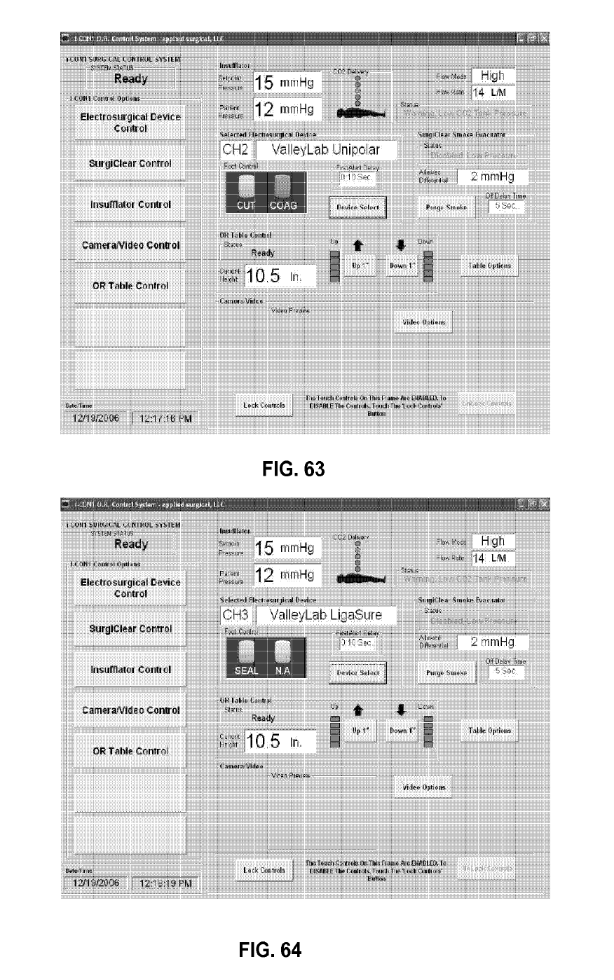

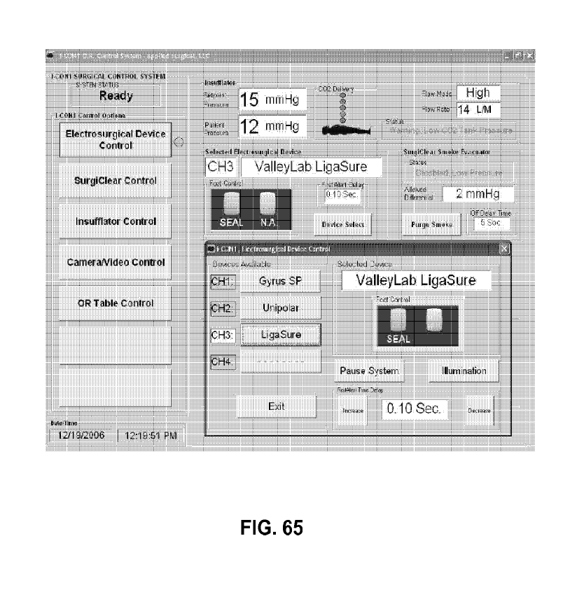

In another aspect, the control system can form a portion of an overall integrated operating room control system. Referring now to FIGS. 61-63, leading hospitals today are purchasing cutting-edge integration technology for both operating efficiency and surgeon and patient recruitment purposes. These integrated OR suites feature a full array of interconnected OR equipment, from peripherals to essentials, with one notable exception—the electrosurgical instruments, which are arguably the most essential of all the tools. The control system 10 provides a key or core component in achieving substantially total OR integration (where multiple ESU manufacturers are concerned). This integrated OR control system could take several forms, with the preferred embodiment comprising the control system with both on/off and linear control capabilities as desired, smoke evacuation control (which can include a deeper integration with an insufflator, allowing for a true closed-loop insufflator/evacuator system), insufflator control, camera/video control, OR table control, and additional peripherals control as desired.

Optionally, the integrated OR control system could comprise the electrosurgical and linear devices, camera/video control, insufflator control, and smoke Evacuation control. Even this simplified integrated form provides much-needed integration of the essentials of the OR but could be sold at a price point that would open up an additional 40-50% of the market that otherwise can not afford the current integrated OR technology (which, incidentally, lacks integration of a number of the essentials).

As persons skilled in the art to which the invention relates understand, the above-described method steps and the software embodying them can be structured and can flow in various ways other than the exemplary structure and flow described above. The software can be modularized or otherwise structured in any suitable manner, with the above-mentioned “routines” and use of interrupts being only one example.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A control system for a plurality of independent electrosurgical devices, wherein the plurality of devices comprises at least one device having a first output function or operating characteristic and least a second device having a second output function or operating characteristic that differs from the first output function or operating characteristic, the control system comprising:
a device selector operable by a user to select one of the electrosurgical devices;
a central user control operable by a user to selectively operate the selected electrosurgical device; wherein the central user control is configured to selectively operate at least one electrosurgical device selected from a group consisting of: a linear type electrosurgical device and an on/off type electrosurgical device; and
a plurality of adaptors, wherein at least one adaptor is configured to couple with the electrosurgical device having the first output function or operating characteristic and wherein at least one adaptor is configured to couple with the electrosurgical device having the second output function or operating characteristic, wherein each adaptor is configured to communicate information regarding the associated electrosurgical device to a processor system that is responsive to operation of the central user control, and wherein the processor system is programmed or configured to respond to operation of the central user control by activating the selected electrosurgical device in accordance with information communicated by the associated adapter.

2. The control system of claim 1, wherein at least one adaptor is removably connectable to the processor system.

3. The control system of claim 2, wherein the adaptor further comprises a cable and a connector for removeable connection to the processor system.

4. The control system of claim 1, wherein the central user control is configured to send a control signal initiating the operation a smoke evacuation system upon selection or activation of an given electrosurgical device.

5. The control system of claim 1, wherein the central user control has a plurality of central user inputs operable by a user to control functions of the electrosurgical devices, wherein each function of an electrosurgical device is associated with one of a plurality of device user inputs of the associated device user control; and the device selector is operable by a user to selectably associate each central user input with one of the functions of a selected electrosurgical device.

6. The control system of claim 5, wherein the plurality of central user inputs comprises a left foot pedal and a right foot pedal; the device selector is operable by a user to selectably associate the left foot pedal with one of the functions of a first electrosurgical device and associate the right foot pedal with one of the functions of a second electrosurgical device.

7. A system, comprising:
a plurality of independent electrosurgical devices, wherein the plurality of devices comprises at least one device having a first output function or operating characteristic and least a second device having a second output function or operating characteristic that differs from the first output function or operating characteristic, wherein the plurality of independent electrosurgical devices is selected from a group consisting of: linear type electrosurgical devices and on/off type electrosurgical devices;
a device selector operable by a user to select one of the electrosurgical devices;
a central user control operable by a user to selectively activate the selected electrosurgical device;
a processor system that is responsive to operation of the central user control; and
a plurality of adaptors, wherein at least one adaptor is configured to couple with the electrosurgical device having the first output function or operating characteristic and wherein at least a second adaptor is configured to couple with the electrosurgical device having the second output function or operating characteristic, wherein each adaptor is configured to communicate information regarding the associated electrosurgical device to the processor system,
wherein the processor system is programmed or configured to respond to operation of the central user control by activating the selected electrosurgical device in accordance with information communicated by the associated adapter.

8. The system of claim 7, wherein at least one adaptor is removably connectable to the processor system.

9. The system of claim 8, wherein the adaptor further comprises a cable and a connector for removeable connection to the processor system.

10. The system of claim 7, wherein: the central user control has a plurality of central user inputs operable by a user to control functions of the electrosurgical devices, wherein each function of an electrosurgical device is associated with one of a plurality of device user inputs of the associated device user control; and the device selector is operable by a user to selectably associate each central user input with one of the functions of a selected electrosurgical device.

11. The system of claim 7, wherein: the device user control associated with each electrosurgical device of the plurality of electrosurgical devices is a foot control; and the central user control is a foot control.

12. The system of claim 11, wherein the foot control is a wireless foot control.

13. The system of claim 7, wherein the communicated information characterizes operation of the device user control associated with the selected electrosurgical device.

14. The system of claim 7, wherein each intelligent adaptor includes a cable with a first connector removably connectable to the associated electrosurgical device and a second connector removably connectable to an enclosure housing the processor system.

15. The system of claim 7, further comprising a display, wherein the processor system is programmed or adapted to display information communicated by the associated intelligent adapter.

16. The system of claim 15, wherein the communicated information identifies a device type of the selected electrosurgical device; and the processor system is programmed or adapted to display an indication of the device type of the selected electrosurgical device.

17. The system of claim 15, wherein the communicated information identifies a manufacturer and model of the selected electrosurgical device; and the processor system is programmed or adapted to display indications of the manufacturer and model of the selected electrosurgical device.

18. The system of claim 15, wherein the communicated information identifies a function of the selected electrosurgical device; and the processor system is programmed or adapted to display an indication of the function of the selected electrosurgical device.

19. The system of claim 15, wherein the communicated information identifies a function of the selected electrosurgical device; the display comprises at least one verification status indicator and at least one verification command button; wherein the processor system is programmed or adapted to display an indication of the device verification status of the selected electrosurgical device.

20. The system of claim 7, wherein the device selector includes a display, and wherein the processor system is programmed or adapted to display information communicated by the associated intelligent adapter.

21. The system of claim 20, wherein the communicated information identifies a device type of the selected electrosurgical device; and the processor system is programmed or adapted to display an indication of the device type of the selected electrosurgical device.

22. The system of claim 21, wherein the communicated information identifies a manufacturer and model of the selected electrosurgical device; and the processor system is programmed or adapted to display indications of the manufacturer and model of the selected electrosurgical device.

23. The system of claim 21, wherein the communicated information identifies a function of the selected electrosurgical device; and the processor system is programmed or adapted to display an indication of the function of the selected electrosurgical device.

24. The system of claim 21, wherein the communicated information identifies a function of the selected electrosurgical device; and the control unit engages a smoke evacuation system upon activation of the electrosurgical device when said function is a surgical function.

25. The system of claim 24, wherein the smoke evacuation system comprises: (a) a smoke evacuator, and (b) an insufflator.

26. The system of claim 24, wherein the smoke evacuation system remains activated for a predetermined amount of time.

27. The system of claim 7, wherein one or more of said intelligent adapters further comprises an adapter module.
